(19) United States
(12) Patent Application Publication
(10) Pub. No.: US 2022/0336110 A1
(43) Pub. Date: Oct. 20, 2022

(54) CASE SIFT AND CLUSTER SIFT FOR OUTBREAK TRACKING AND MANAGEMENT

(71) Applicants: BAXTER INTERNATIONAL INC., Deerfield, IL (US); BAXTER HEALTHCARE SA, Glattpark (Opfikon) (CH)

(72) Inventors: Paul Randall, Herefordshire (GB); Chinemerem Eyetan, Milton Keynes (GB); Sam Wilkinson, Gloucester (GB)

(21) Appl. No.: 17/724,592

(22) Filed: Apr. 20, 2022

Related U.S. Application Data
(60) Provisional application No. 63/177,126, filed on Apr. 20, 2021.

(51) Int. Cl. G16H 50/80; G06N 5/02; G16H 50/30; G16H 10/60

(52) U.S. Cl. CPC G16H 50/80; G06N 5/025; G16H 50/30; G16H 10/60

(57) ABSTRACT

A graph database for outbreak tracking and management is disclosed. In an example, an outbreak management system includes a memory device storing instructions that define a graph database for disease outbreak tracking. The instructions specify, for a given host, that a host node is created and an episode node is connected to the host node via a ‘case’ link. Additionally, the instructions specify that a rule node is connected to the episode node via a ‘found’ link. The rule node is associated with a workflow, an assignment, and a notification, and includes at least one defined condition. If a condition is satisfied for a case, the rule node is linked to the episode node. This leakage causes the workflow to be added for the episode node, the episode node to be assigned to an individual or a team, and a notification to be generated regarding an association with the rule.

### PRIORITY CLAIM

This application claims priority to and the benefit as a non-provisional application of U.S. Provisional Patent Application No. 63/177,126, filed on Apr. 20, 2021, the entire contents of which are hereby incorporated by reference and relied upon.

### BACKGROUND

As of spring 2020, the world population was approximately 7.7 billion people. The United Nations estimates that the population will reach 8 billion by 2024 and 9 billion by 2042. As the world's population increases, the population is relocating to more dense suburban and urban areas in addition to encroaching upon undeveloped areas, such as forests and grasslands. For example, the city of Manila in the Philippines has a density of 107,000 people per square mile while more populous Indian cities, such as Mumbai have almost 12 million people with an average density of 73,000 people per square mile.

In addition to the population increasing and becoming denser, travel, such as airline travel, is becoming more commoditized, enabling more people to travel further distances. Increasing wages among the world's middle class enables more of the world's population to travel regionally, nationally, or internationally. Globalization of business and trade further increases daily foreign contacts. Altogether, increased populations living in dense areas combined with an increasingly mobile population creates optimal conditions for the rapid spread of contagions and other outbreaks. These optimal conditions helped turn the severe acute respiratory syndrome (“SARS”) CoV-2 (“COVID-19”) into a worldwide pandemic during 2020 and 2021. Before COVID-19, the world had a number of scares including other SARS viruses, swine flu, and Ebola. The Center for Disease Control and Prevention (“CDC”) evens maintains a website of currently recognized outbreaks in the United States and worldwide.

While the world advances, the tracking and management of outbreaks lags behind. This was especially evident during the tracking of the spread of COVID-19. Oftentimes, it takes disease researchers weeks to months to identify an outbreak and even longer to determine its distribution, spread, and origin. Generally, infection tracking is a manual process that involves studying medical records, government records, field notes, and using an army of medical workers to perform contact tracing. Some government organizations and large medical centers build and analyze relational databases of outbreak information to determine distribution, spread, and origin. However, extensive effort is needed to input the correct data. Additionally, significant computational power is needed to analyze the collected data. As a result of the effort and power needed, only relatively severe or harmful outbreaks are tracked, leaving many outbreaks untracked.

In addition to above, medical data related to an outbreak is only entered after an outbreak has been identified. The lag between the outbreak occurring, identification, data gathering, and analysis can result in an outbreak quickly expanding before much is known. As many healthcare professionals believe, the next pandemic (or wave of pandemics) will begin small, in isolated areas such as urban centers or on the fringes of developed areas, and proceed under health radars until a significant portion of the world's population is affected.

### SUMMARY

The present disclosure sets forth a graph database for outbreak tracking and management. In particular, the disclosure describes a method, system, and apparatus for the creation and updating of a graph database to track one or more outbreaks. The method, system, and apparatus disclosed herein may also be configured to compare certain graph database data, patient data, and/or patient medical data to one or more case or cluster rules. Satisfaction of the case or cluster rule causes the method, system, and apparatus to add a case to an outbreak, begin a workflow to ensure the case is properly documented or otherwise managed, and/or transmit notifications to designated clinicians indicative of a presence of a new case. The graph databases incorporate medical, positional, temporal, and/or demographic relationships between nodes according to a predefined structure. The definitions and relationships provided among different types of nodes enable information to automatically be incorporated into the graph database. The disclosed graph database structure enables cases to be analyzed to determine if an outbreak is in the process of occurring. Linkage of cases to an outbreak enable an origin of the outbreak to be tracked to help identify potential solutions and appropriate control measures.

Aspects of the subject matter described herein may be useful alone or in combination with one or more other aspects described herein. Without limiting the foregoing description, in a first aspect of the present disclosure, an outbreak management server apparatus includes an outbreak management server system including a memory device storing (i) case rules each specifying at least one condition for satisfying the respective case rule, and (ii) instructions therein. The instructions define a graph database structure for disease outbreak tracking. The instructions specify for a given host that a host node is created, the host node being associated with host parameters, an episode node is connected to the host node via a ‘case’ link, the episode node being associated with episode parameters that are related to a disease classification of the host, and a rule node is connected to the episode node via a ‘found’ link, the rule node being associated with a case workflow, an assignment, and a notification. The outbreak management server apparatus also includes an outbreak management server configured to receive patient data related to the host and store at least some of the received patient data to the graph database at one or more parameters of at least one of the host node or the episode node based on contents of the at least some of the received patient data matching parameter definitions of the respective node. The outbreak management server apparatus is also configured to compare parameters of the host node and episode node to the conditions of the case rules, and for a case rule having at least one condition that first matches the parameters of the host node and episode node, create a link between the rule node and the episode node, the linkage causing a workflow of the case rule to be added for the episode node, the episode node to be assigned to an individual or a team, and a notification to be generated regarding association with the case rule.

In a second aspect of the present disclosure, which may be combined with any other aspect, or portion thereof, described herein, the case rules are arranged in an order or hierarchy, and the outbreak management server is further configured to sequentially compare the one or more conditions of each of the case rules to the parameters of the host node and episode node until there is a match to one case rule.

In a third aspect of the present disclosure, which may be combined with any other aspect, or portion thereof, described herein, the case workflow defines a sequence of steps specified by objects for obtaining documentation related to the case of the episode node or managing a disease associated with the episode node.

In a fourth aspect of the present disclosure, which may be combined with any other aspect, or portion thereof, described herein, the outbreak management server is further configured to use the assignment of the case rule to create a leakage between the episode node and a node of an individual or a team via an ‘assigned’ link.

In a fifth aspect of the present disclosure, which may be combined with any other aspect, or portion thereof, described herein, the notification identifies one or more individuals or organizations that should receive a message indicative of the case rule being satisfied for the host node and the episode node.

In a sixth aspect of the present disclosure, which may be combined with any other aspect, or portion thereof, described herein, the case rule includes guidance information for treating the case, the guidance information being included within the message that is related to the notification.

In a seventh aspect of the present disclosure, which may be combined with any other aspect, or portion thereof, described herein, each of the case rules are data structures or files that include the one or more conditions, assignment information, a link to the case workflow, and notification information.

In an eighth aspect of the present disclosure, which may be combined with any other aspect, or portion thereof, described herein, after the case workflow is completed, the outbreak management server is further configured to connect an outbreak node to the episode node via a ‘part of’ link to indicate that the host has become part of an outbreak of the disease when a result of the workflow is indicative that the case is associated with the outbreak. The outbreak node is connected to a definition node via a ‘defined as’ link, the definition node specifying disease parameters of the disease that are related to the outbreak node.

In a ninth aspect of the present disclosure, which may be combined with any other aspect, or portion thereof, described herein, the disease parameters include at least one of a name of the disease, a background of the disease, a time/place related to the disease, clinical criteria for the disease, laboratory criteria for the disease, modes of transmission for the disease, criteria for determining a ‘suspected’ classification, criteria for determining a ‘probable’ classification, and criteria a determining the ‘confirmed’ classification.

In a tenth aspect of the present disclosure, which may be combined with any other aspect, or portion thereof, described herein, the server is further configured to connect the host node to the outbreak node via the episode node after determining at least some of the patient data matches at least some of the disease parameters of the definition node.

In an eleventh aspect of the present disclosure, which may be combined with any other aspect, or portion thereof, described herein, the host parameters include at least one of a name of the host, a patient classification flag, a clinician classification flag, a person classification flag, an animal classification flag, a fomite classification flag, an object classification flag, patient demographic data, or patient medical data.

In a twelfth aspect of the present disclosure, which may be combined with any other aspect, or portion thereof, described herein, the episode parameters include at least one of a start of a case number for the host, a ‘possible’ classification for the disease for the host, a ‘probable’ classification for the disease for the host, a ‘confirmed’ classification for the disease for the host, an immunization status of the host, an immunization type of the host, a flag indicative that the host acquired the disease in a medical facility, or death information related to the host.

In a thirteenth aspect of the present disclosure, which may be combined with any other aspect, or portion thereof, described herein, the outbreak management server is further configured to generate a case number for the respective episode parameter of the episode node after determining a ‘confirmed’ or a ‘probable’ classification for the host.

In a fourteenth aspect of the present disclosure, which may be combined with any other aspect, or portion thereof, described herein, the outbreak management server is further configured to receive at least one of a geographic location or a journey associated with the host node, create an activity map that is related to the host node for the at least one geographic location or journey, and create stay nodes linked to the host node via a ‘stayed’ link and location nodes linked to one of the stay nodes via an ‘in’ link for the at least one geographic location or journey.

In a fifteenth aspect of the present disclosure, which may be combined with any other aspect, or portion thereof, described herein, an outbreak management method includes storing, in a memory device, (i) case rules each specifying at least one condition for satisfying the respective case rule, and (ii) a graph database structure for disease outbreak tracking, the graph database structure configured, for a given host, such that when a host node is created, the host node is associated with host parameters, when an episode node is connected to the host node via a ‘case’ link, the episode node is associated with episode parameters that are related to a disease classification of the host, and when a rule node is connected to the episode node via a ‘found’ link, the rule node is associated with a case workflow, an assignment, and a notification. The method also includes receiving, in an outbreak management server, patient data related to the host, storing, via the outbreak management server, at least some of the received patient data to the graph database at one or more parameters of at least one of the host node or the episode node based on contents of the at least some of the received patient data matching parameter definitions of the respective node, comparing, via the outbreak management server, parameters of the host node and the episode node to the conditions of the case rules, and for a case rule having at least one condition that first matches the parameters of the host node and episode node, creating, via the outbreak management server, a link between the rule node and the episode node, the linkage causing a case workflow of the case rule to be added for the episode node, the episode node to be assigned to an individual or a team, and a notification to be generated regarding association with the case rule.

In a sixteenth aspect of the present disclosure, which may be combined with any other aspect, or portion thereof, described herein, the case rules are arranged in an order or hierarchy, and the method further comprises sequentially comparing, via the outbreak management server, the one or more conditions of each of the case rules to the parameters of the host node and episode node until there is a match to one case rule.

In a seventeenth aspect of the present disclosure, which may be combined with any other aspect, or portion thereof, described herein, the case workflow defines a sequence of steps specified by objects for obtaining documentation related to the case of the episode node or managing a disease associated with the episode node.

In an eighteenth aspect of the present disclosure, which may be combined with any other aspect, or portion thereof, described herein, the method further comprises using, via the outbreak management server, the assignment of the case rule to create a leakage between the episode node and a node of an individual or a team via an ‘assigned’ link.

In a nineteenth aspect of the present disclosure, which may be combined with any other aspect, or portion thereof, described herein, the notification identifies one or more individuals or organizations that should receive a message indicative of the case rule being satisfied for the host node and the episode node, and the case rule includes guidance information for treating the case, the guidance information being included within the message that is related to the notification.

In a twentieth aspect of the present disclosure, which may be combined with any other aspect, or portion thereof, described herein, the method further includes connecting, via the outbreak management server, an outbreak node to the episode node via a ‘part of’ link to indicate that the host has become part of an outbreak of the disease when a result of the workflow is indicative that the case is associated with the outbreak. The outbreak node is connected to a definition node via a ‘defined as’ link, the definition node specifying disease parameters of the disease that are related to the outbreak node.

In accordance with a twenty-first aspect of the present disclosure, any of the structure and functionality illustrated and described in connection with FIGS. 1 to 32 may be used in combination with any of the structure and functionality illustrated and described in connection with any of the other of FIGS. 1 to 32 and with any one or more of the preceding aspects.

In light of the aspects above and the disclosure herein, it is accordingly an advantage of the present disclosure to provide graph database with case rules that define workflows, assignments, and notifications that are added to each case.

It is another advantage of the present disclosure to provide a system that creates graph databases according to a predefined data structure to enable a source of an outbreak to be determined using one or more care or cluster rules.

It is further advantage of the present disclosure to provide a system that creates graph databases according to a predefined data structure to identify potential or susceptible individuals to an outbreak.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIGS. 1 to 3 show example embodiments of an example outbreak tracking and management system including an outbreak management server, according to example embodiments of the present disclosure.

FIG. 4 shows a diagram of the outbreak management server of FIGS. 1 to 3, according to an example embodiment of the present disclosure.

FIG. 5 shows a diagram of an example patient electronic medical record, according to an example embodiment of the present disclosure.

FIGS. 6 and 7 show diagrams of graphical representations of outbreak conditions, according to example embodiments of the present disclosure.

FIG. 8 shows a diagram of an outbreak graph database structure, according to an example embodiment of the present disclosure.

FIG. 9 shows a diagram of an interface screen of an outbreak graph database for a specified Host Node, according to an example embodiment of the present disclosure.

FIG. 10A shows a diagram of a hierarchical location map for use with a graph database, according to an example embodiment of the present disclosure.

FIG. 10B shows a diagram of a graph database that includes Specimen Nodes, Isolate Nodes, and Organism Nodes, according to an example embodiment of the present disclosure.

FIG. 11 shows a diagram of an example graph database for a Ward Location Node, according to an example embodiment of the present disclosure.

FIG. 12 shows a diagram of an example graph database for an Influenza Outbreak Node, according to an example embodiment of the present disclosure.

FIGS. 13 to 18 show diagrams of example interface screens of outbreak analysis data determined from an outbreak graph database, according to an example embodiment of the present disclosure.

FIGS. 19 and 20 illustrate flow diagrams showing example procedures to configure and create graph databases, according to example embodiments of the present disclosure.

FIG. 21 illustrates a flow diagram showing an example procedure for creating one or more rules for case and/or cluster sifting for outbreak classification, according to an example embodiment of the present disclosure.

FIG. 22 is a diagram of a user interface for specifying a case and/or cluster rule, according to an example embodiment of the present disclosure.

FIG. 23 is a diagram of a user interface for entering condition information, according to an example embodiment of the present disclosure.

FIG. 24 shows a user interface provided by the outbreak management server of FIG. 1 for configuring case information for population into the user interface of FIG. 22, according to an example embodiment of the present disclosure.

FIG. 25 is a diagram of a user interface for a custom workflow, according to an example embodiment of the present disclosure.

FIG. 26 is a diagram of a case/cluster user interface provided by the outbreak management server of FIG. 2 for display in an application of a user device, according to an example embodiment of the present disclosure.

FIG. 27 is a diagram of another case/cluster user interface provided by the outbreak management server of FIG. 2 for display in the application of the user device, according to an example embodiment of the present disclosure.

FIG. 28 illustrates a flow diagram showing an example procedure for comparing one or more cases to one or more case and/or cluster rules, according to an example embodiment of the present disclosure.

FIG. 29 is a diagram of a graph database including a link between an episode node of a case to a matching rule, according to an example embodiment of the present disclosure.

FIGS. 30 to 32 are diagrams of user interfaces for activity mapping, according to an example embodiment of the present disclosure.

### DETAILED DESCRIPTION

The present disclosure relates in general to a method, system, and apparatus that create and analyze outbreak graph databases for outbreak management. The graph databases disclosed herein are specifically configured to provide a medical, positional, and/or temporal relationship between components of an outbreak. The unique definition between the components of the graph database enable links to be automatically determined among a plurality of individuals with minimal input from clinicians. The example method, system, and apparatus disclosed herein are configured to perform certain defined analytics on the graph database to determine, in near real-time, distribution, spread, and origin of an outbreak as soon as a first suspected, probable or confirmed case is received. The example method, system, and apparatus may also apply one or more rules to cases or a cluster of cases to determine if the data is indicative of an outbreak. The rules may define one or more workflows for processing a case and obtaining sufficient documentation for disease classification.

In comparison to graph databases, traditional relational databases comprise tables of data. Unique keys are used to link certain data together from different tables. Relational databases are generally adapted for flat data layouts, where relationships between data are only one to three levels deep. As additional data levels are added or data becomes increasing interrelated, relational databases require significant computational power for analysis. Some computation may be avoided using indexes with relational databases. However, the indexes may be stale as new data is added, which may require recompilation of the databases and subsequent analysis.

As disclosed herein, the methods, apparatus, and system are configured to operate with graph databases for outbreak management. A graph database includes a data structure that connects or links nodes via a data relationship (e.g., an edge). The relationships between nodes may be semantic, which enable semantic analysis and queries to be conducted. Each of the nodes may have one or more parameters or attributes that define and store underlying data. The example graph database disclosed herein enables complex hierarchical structures to be computationally efficiently modeled and constantly updated as new data is provided. In some instances, the disclosed graph databases may have seven to twenty different data levels. Graph databases are especially well adapted for outbreak management since the hierarchical multi-level nature of a graph database approximates the actual spread of a condition during an outbreak.

The graph database disclosed herein includes nodes and edges/relationships. The graph database is configured to relate data items in a memory to a collection of nodes and edges. In some embodiments, the nodes and edges may be stored in a table or list, with edges being used to link nodes together. The edges of a graph database enable stored data to be linked together and retrieved with one or a few operations. In the illustrated example, a node represents an outbreak, a definition of an outbreak, an episode of an outbreak, a host (e.g., a person, object, an animal, equipment, a fomite, etc.), a symptom of a host, a location of a host, a date/time a host was at a location, and a location hierarchy (e.g., organizational structure of a hospital facility). Each node has one or more parameters or attributes, which define stored data that is relevant to the node. Nodes also include edges or relationships. The edges or relationships connect nodes together and define the relation between them.

The example graph databases created and processed by the example methods, apparatus, and system may be stored in a data structure configured for storing nodes, parameters or attributes, and relationships. In some embodiments, the graph database may be configured for a query language such as Gremlin, SPARQL, Cypher, etc. In other embodiments, the graph database may be accessed via one or more application programming interfaces (“APIs”).

The example graph databases disclosed herein enable outbreak information to be created automatically from patient medical records, demographic databases, and/or clinician entry information. As such, the graph databases enable outbreaks to be tracked for each person identified as having a defined condition. As additional information is received, the example system, methods, and apparatus are configured to update, in near-real time, the graph database to provide an up-to-date representation of potential and actual outbreaks. Additional information is easily added by the example system, methods, and apparatus through the creation of nodes, parameters or attributes, and/or relationships. Since the new nodes, parameters or attributes, and relationships build on what is already in the graph database, re-compilation of the database is not needed.

The example system, method, and apparatus disclosed herein are configured to analyze the graph databases to determine the distribution, spread, and origin of an outbreak in addition to providing information to help stop the spread. For example, the system, method, and apparatus disclosed herein may be configured to identify ‘at risk’ persons for quarantine or vaccination, provide graphical representations to illustrate the progress of an outbreak, identify points of intervention, provide reports to governmental bodies, conduct statistical analysis of outbreaks to determine for example, attack rates, epicurves, etc., and/or combine outbreak data to determine an overall picture of a burden of an outbreak on a health system region, or country.

The example system, method, and apparatus disclosed herein are configured to create and manage different types of outbreaks. As disclosed herein, outbreaks may include small public health incidents (e.g., food poisoning) and/or large public health incidents (e.g., epidemics or pandemics). Outbreaks may also include traumatic neurological events, contamination events, and/or hospital-based contagion infections. As such, outbreaks are not limited to viral or bacterial infections, but can also include chemical, radiation, or biological exposures.

I. Outbreak Tracking and Management System Embodiments

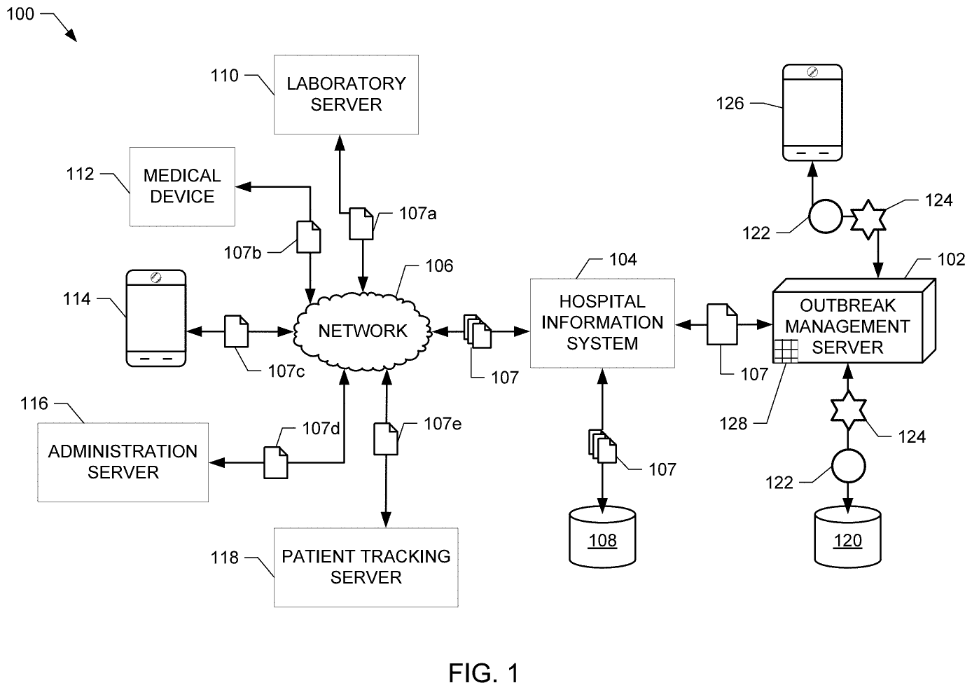

FIGS. 1 to 3 show example embodiments of an example outbreak tracking and management system 100, according to example embodiments of the present disclosure. The example system 100 includes an outbreak management server 102 and a Hospital Information System (“HIS”) 104. The server 102 is configured to create, analyze, and manage graph databases. The example HIS 104 is configured to provide connectivity within a hospital or medical environment. In some examples, the server 102 may be included within the HIS 104. In other examples, the server 102 may be external to and communicatively coupled to the HIS 104.

The example HIS 104 is communicatively coupled via a network 106 to one or more devices or computers that are configured to transmit or otherwise provide patient medical data 107. The HIS 104 stores the patient medical data 107 as an Electronic Medical Record (“EMR”) within a memory device 108. In the illustrated example, the HIS 104 is communicatively coupled to a laboratory server 110, which is configured to transmit laboratory patient medical data 107a, a medical device 112 configured to transmit device patient medical data 107b, a clinician device 114 configured to transmit clinician patient medical data 107c, an administration server 116 configured to transmit administration patient medical data 107d, and a patient tracking server 118 configured to transmit patient tracking medical data 107e.

The laboratory server 110 may be communicatively coupled to one or more laboratory instruments that generate laboratory data from analysis of one or more biological samples from a patient. The laboratory server 110 stores the laboratory data as the laboratory patient medical data 107a, which is periodically transmitted to the patient's EMR via the HIS 104, which is stored at the memory device 108.

The medical device 112 includes any type of clinical medical device including an infusion pump, a renal failure therapy machine, a physiological sensor, a patient bedside monitor, a pulse-ox monitor, a CT scanner, an MRI scanner, etc. The medical device 112 generates operational data and/or alarms/alerts regarding a treatment performed on a patient or a measurement performed on a patient. The data is stored as the device patient medical data 107b and transmitted to the patient's EMR located at the memory device 108. While the example environment 100 of FIG. 1 shows one medical device, it should be appreciated that the environment 100 may include tens to thousands of medical devices.

The example clinician device 114 includes any smartphone, tablet computer, laptop computer, desktop computer, workstation, etc. that is configured to receive clinician-entered data regarding a condition of a patient. The data may include observation notes, prescriptions, treatments, diagnosis, observed/identified symptoms, etc. The received data is stored as the clinician patient medical data 107c and transmitted to the patient's EMR, which is stored at the memory device 108. While the example environment 100 of FIG. 1 shows one clinician device 114, it should be appreciated that the environment 100 may include tens to thousands of devices.

The example administration server 116 is configured to receive patient administration information. The information includes patient demographic and/or physiological information such as gender, weight, age, birth date, height, medical history, etc. The information may also include a ward (e.g., care area) and/or room assigned to the patient. The information may be received at the time of admittance of the patient or entered/acquired after a patient has been admitted or moved to a new location in a medical facility. The administration server 116 transmits the administration information as the administration patient medical data 107d to the patient's EMR at the memory device 108.

The example patient tracking server 118 is configured to track a patient's location in a medical facility. The example server 118 is configured to receive information indicative of a ward to which a patient is assigned or has been moved. The server 118 is also configured to receive information indicative of a room or bed to which the patient is assigned. The server 118 transmits the patient tracking medical data 107e to the patient's EMR at the memory device 108 via the HIS 104. The server 118 may transmit the data 107e each time a patient's location changes, and may include an indication of a discharge.

The example network 106 may include any wired or wireless local area network (“LAN”) and/or wide area network (“WAN”). In some examples, the network 106 may include one or more firewalls, gateways, and/or switches that control access, formatting, and data routing. The network 106 may be configured to be self-contained within a medical facility and/or may include an external network and corresponding interfaces/virtual tunnels.

The example HIS 104 is configured to store the received data 107 to the appropriate patient EMR stored in the memory device 108. In some embodiments, the patient medical data 107 includes a patient name or other identifier that corresponds to an identifier within or linked to the EMR. The HIS 104 compares identifiers to determine a match. After a match is identified, the HIS 104 stores the patient medical data to the matching EMR.

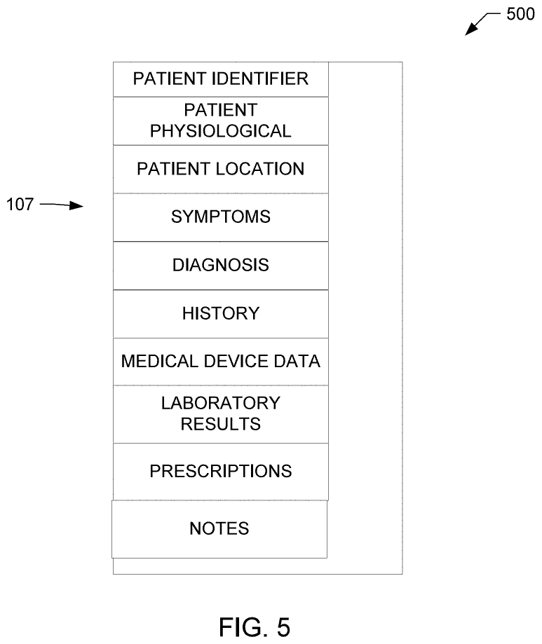

FIG. 5 shows an example patient EMR 500, according to an example embodiment of the present disclosure. The EMR 500 includes patient medical data 107 received from one or more of the devices 110 to 118 of FIG. 1. This includes a patient identifier and/or name, patient physiological and/or demographic information, patient location(s), symptoms, diagnosis, medical history, medical device data, laboratory results, prescriptions, and notes. The patient medical data 107 is stored in a relational or table data structure and provides a substantive medical characterization of a patient. In some examples, the different categories of information include data fields, labels, or metadata that identifies the stored data. For example, the EMR 500 may include a label “room number” next to an alphanumeric value that corresponds to a patient's room and bed. In other embodiments, each of the different types of patient medical data are stored to different fields or sections of the EMR 500 in a predefined or pre-structured arrangement.

Returning to FIG. 1, the example HIS 104 is configured to provide access to the patient EMRs 500 (and more generally the patient medical data 107) that is located in the memory device 108. For example, the clinician device 114 may access the memory device 108 via the HIS 104 to view, edit, add, or remove patient medical data from a patient's EMR. In addition, the example outbreak management server 102 is configured to periodically (e.g., every 60 seconds, every 5 minutes, every hour, etc.) or continuously access the memory device 108 to acquire patient medical data 107. Additionally or alternatively, the HIS 104 may transmit a copy of patient medical data 107 to the outbreak management server 102 at periodic times or as the data is received.

The example outbreak management server 102 is configured to enable clinicians to specify a framework for the creation of outbreak graph databases. The example outbreak management server 102 operates accordingly to the framework to automatically (or with minimal clinician input) create graph databases for specified types of outbreaks. The outbreak management server 102 is also configured to analyze graph databases to determine a distribution, spread, and origin of outbreaks. The management server 102 is configured to render graph databases into a graphical representation to provide different views of an outbreak or provide results of an analysis or semantic query. The outbreak management server 102 may be configured to provide an interactive graphical representation that enable a clinician to filter or hide certain levels of data or view parameter or attribute values of the underlying data for one or more specified nodes.

The outbreak management server 102 is communicatively coupled to a memory device 120, which is configured to store graph databases. As illustrated in FIG. 1, the outbreak management server 102 receives or otherwise accesses copies of patient medial data 107. The outbreak management server 102 is configured to create, from the copy of the patient medical data 107, graph databases 122, including nodes, relationships, and parameters of graph databases. The outbreak management server 102 analyzes the graph databases 122 to create analysis data 124, which is also stored in the memory device 120.

The outbreak management server 102 of FIG. 1 is communicatively coupled to one or more user devices 126 via a wired or wireless network. The server 102 is configured to transmit the graph databases 122 and/or analysis data from the memory device 120 for viewing, navigating, and/or editing at the user device 126. In some embodiments, the user device 126 accesses the outbreak management server 102, which provides and/or transmits an interface for interacting with the graph databases 122 that are located in the memory device 120. The interface is configured to include features that enable a user to submit a semantic query and/or select options for analysis. In response to the request from the user device 126, the example outbreak management server 102 performs the requested analysis on the graph database to generate the analysis data 124, which is sent to the user device 126 for display. The interface may also be configured to enable the user device 126 to modify or add nodes, relationships, and/or parameters to a graph database (or confirm nodes, relationships, and/or parameters).

The example user device 126 includes a smartphone, tablet computer, laptop computer, desktop computer, workstation, server, etc. In some examples, the user device 126 may comprise the clinician device 114. In other examples, the user device 126 may be a device that is external or separate from a hospital system that can connect to the server 102 via a secure gateway, access port, and/or firewall.

In some embodiments, the outbreak management server 102 operates according to instructions 128 stored in a memory (e.g., the memory device 120), which when executed, cause the outbreak management server 102 to perform the operations, steps, methods, procedures, routines, algorithms, etc. described herein. For example, the instructions enable the server 102 to create, manage, and analyze the outbreak graph databases 122 according to user-specified criteria. The example instructions 128 may also be configured to cause the outbreak management server 102 to improve upon how outbreak medical information is structured in a database by creating a storage structure or framework based on nodes and relationships that approximates actual outbreaks. The instructions 128 specify the creation of outbreak graph databases with well defined relationships between different types of nodes at different data levels, which enables computationally efficient processing and analysis for real-time analysis results and almost instant query results based on semantic language inputs. Further, the example instructions 128 are configured to render and process the graph databases 122 in graphical representations that approximate the actual underlying data structure, which are relatively easy for a user to understand compared to excess quantities of raw patient medical data or data stored in relational databases.

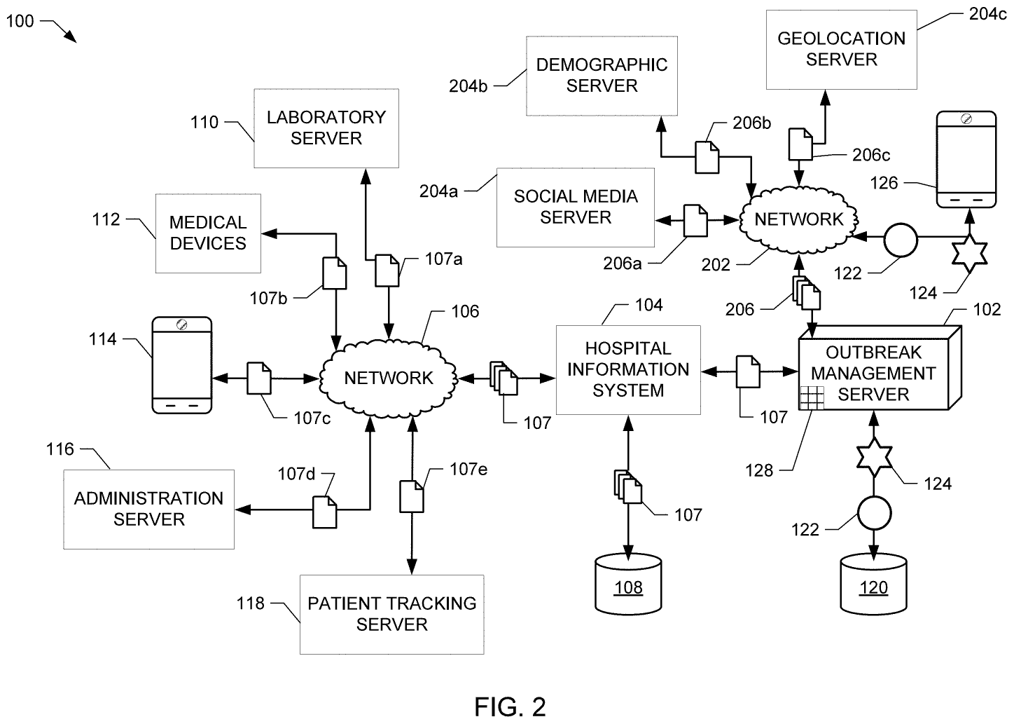

FIG. 2 illustrates a diagram of another embodiment of the outbreak tracking and management system 100 of FIG. 1, according to an example embodiment of the present disclosure. In the illustrated embodiment, the example outbreak management server 102 is communicatively coupled to the user device 126 via a network 202 (e.g., the Internet). The outbreak management server 102 is also communicatively coupled to data servers 204, which are configured to transmit patient data 206 for creating graph databases. The example data servers 204 are configured to generate certain data that describes a location of a patient at a particular date/time. The data servers 204 may also include data that is indicative of a patient's symptoms, physiological information, or anything else that may be relevant for determining nodes, parameters, and/or relationships for a graph database. For example, the social media server 204a is configured to transmit social media data 206a related to a patient. The social media data may include posts, tweets, images, check-in information, etc. The social media data 206a is processed by the outbreak management server 102 using, for example, word maps, keyword identifiers, and other textural natural language search routines to identify data for one or more nodes, relationships, and/or parameters. For example, a social media post may include information indicative that a patient checked into a certain location at a specified time/date. The example outbreak management server 102 is configured to parse the post for the location information for creating a location node. Further, the outbreak management server 102 parses the data 206a for the time/date for a time/date node. The outbreak management server 102 may then create relationships between a node for the patient and the location and times/date nodes, which would be stored in a graph database as: “Person (node)-STAYED (relationship)-Stay (node with start and end date/time)-IN-Location (node)”. In other examples, the server 102 is configured to identify tags within the data 206 to identify contacts of a patient with other individuals at a certain location on a certain date/time.

The example outbreak management server 102 is also communicatively coupled to a demographic server 204b, which is configured to manage demographic information of patients and family relationships. For example, some governmental organizations maintain one or more databases of individuals, which may include the person's name, address, age, gender, race, ethnicity, etc. The databases may also include a list of other individuals that reside at the same address or are related to the person. The server 102 is configured to receive demographic data 206b from the server 204b. The server 102 analyzes the demographic data to create relationships among individuals as well as population parameter information for a patient.

The example outbreak management server 102 is further communicatively coupled to a geolocation server 204c, which is configured to transmit geolocation tracking data. For instance, cellular operators provide location tracking services for smartphones. The operators maintain a database that correlates a user's location (e.g., a GPS location) to a date/time the user was at the location. The server 102 receives this location data 206c from the geolocation server 204c, which it uses to create location and/or date/time nodes regarding the travels of a patient.

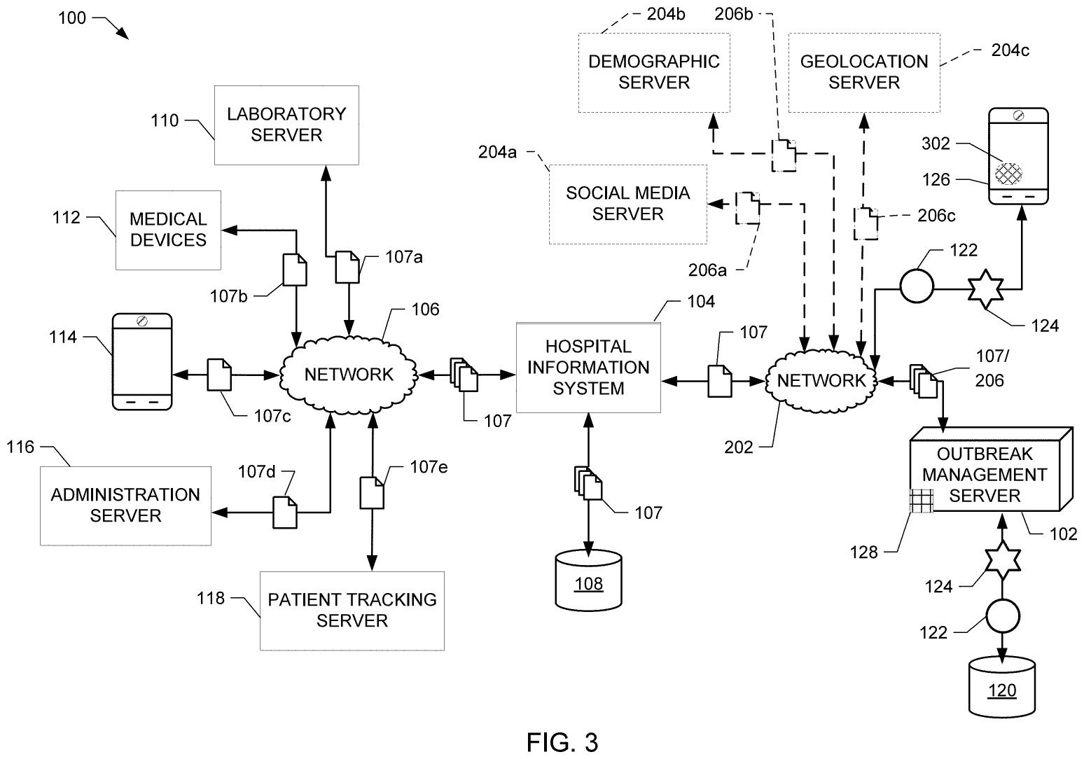

FIG. 3 illustrates a diagram of a further embodiment of the outbreak tracking and management system 100 of FIG. 1, according to an example embodiment of the present disclosure. In the illustrated embodiment, the outbreak management server 102 optionally receives the patient data 206 from the servers 204. In addition, the outbreak management server 102 is communicatively coupled to the HIS 104 via the network 202. In the illustrated embodiment, the server 102 may comprise a cloud-based service host that is configured to provide distributive computing across one or more locations.

FIG. 3 also shows that the user device 126 includes an application 302 (e.g., an App) that is configured to display the graph databases 122. The application 302 is also configured to enable a user of the device 126 to interact with and/or modify the graph database 122 and/or analysis data 124. The application 302 may comprise instructions that cause the device 126 to communicate with one or more APIs at the server 102 for accessing the graph databases 122 and/or the analysis data 124. The application 302 may also include instructions that specify how the graph databases 122 and/or analysis data 124 is to be rendered and displayed on the device 126. The application 302 may further include instructions that define interface tools that a user may use to modify or manipulate the graph databases 122 and/or analysis data 124. In some embodiments, the application 302 may be configured to access the patient medical data 107 at the memory device 108 via the HIS 104.

II. Outbreak Management Server Embodiment

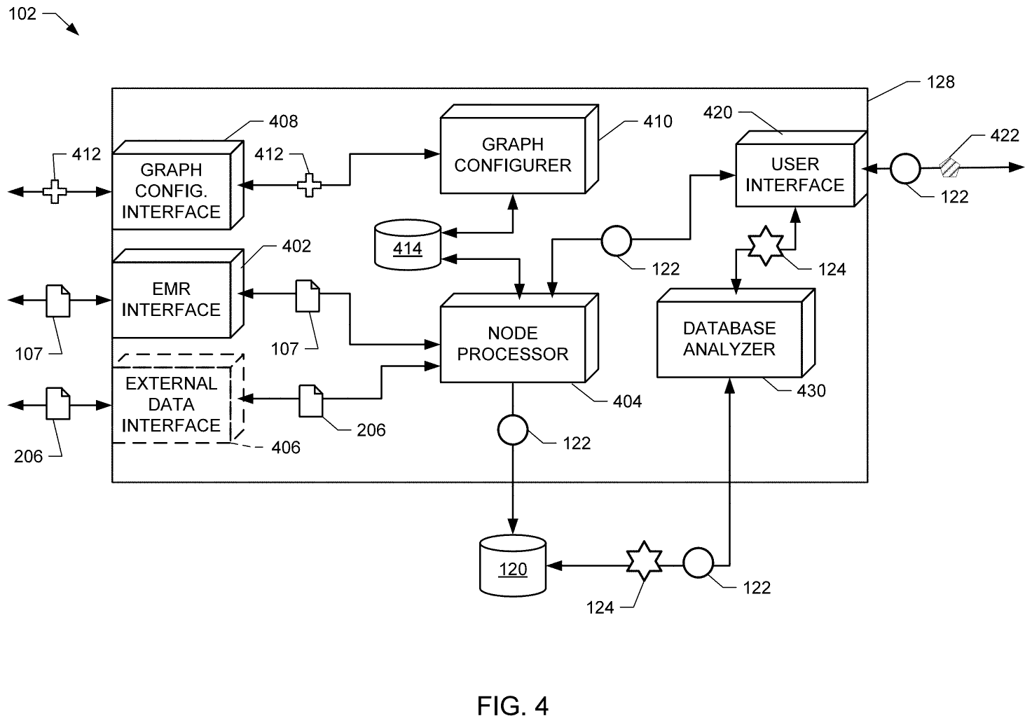

FIG. 4 illustrates a diagram of the outbreak management server 102 of FIGS. 1 to 3, according to an example embodiment of the present disclosure. As disclosed above, operations of the outbreak management server 102 may be defined by the instructions 128 stored in a memory device that is communicatively coupled to the server 102. FIG. 4 shows a graphical representation of the instructions 128 as operational blocks. In some embodiments, the blocks may be combined, added, removed, or further subdivided. It should be appreciated that the graphical representation of the instructions 128 is provided to describe the operations of the server 102. Further, in some embodiments, the instructions 128 may be embodied as hardware or a combination of hardware and software, such as an Application Specific Integrated Circuit (“ASIC”), a microcontroller, and/or a processor. Additionally or alternatively, the instructions 128 may be executed by a single processor or a group of processors, such as in a distributed computing environment.

The example outbreak management server 102 of FIG. 4 includes an EMR interface 402 configured to receive or otherwise acquire a copy of the patient medical data 107 from the HIS 104. The example EMR interface 402 may include one or more instructions for accessing one or more APIs at the HIS 104 for reading the patient medical data 107 from the memory device 108. The interface 402 may additionally or alternatively be configured to transmit request messages for the patient medical data 107. In some instances, the request messages may subscribe to the patient medical data 107 such that changes to the data 107 are automatically sent from the HIS 104 to the EMR interface 402. In some embodiments, the interface 402 may periodically or continually receive the patient medical data 107 from the HIS 104 without having to send a request message.

The example interface 402 is configured to transmit received data 107 to a node processor 404. In some embodiments, the interface 402 may queue the data 107 until the node processor 404 is available. Further, in some embodiments, the interface 402 may be configured to convert the patient medical data from a first format into a second format compatible for processing by the node processor 404 or storage in a graph database. For example, the interface 402 may be configured to convert patient medical data 107 from an HL7 format to an ASCII format for storing data to an EMR or graph database. In some embodiments, the interface 402 converts the data 107 into a JSON, HTML, text, or nonSQL, format.

An external data interface 406 of the server 102 is configured to process the patient data 206 from the external third-party servers 204. Similar to the EMR interface 402, the external data interface 406 may include one or more instructions for accessing one or more APIs at the servers 204 for reading the patient data 206. The interface 406 may additionally or alternatively be configured to transmit request messages for the patient data 206. The request messages may include authentication information to access the patient data 206. The request messages may also include identifiers (e.g., a name, address, alpha-numeric code, etc.) of a patient for which information is being requested. For example, the external data interface 406 may only request information after a host node in an outbreak graph database has been created for a patient based on patient medical data 107. In some instances, the request messages may subscribe to the patient data 206 such that changes to the data are automatically sent from the servers 204 to the external data interface 406. In some embodiments, the interface 406 may periodically or continually receive the patient data 206 from the servers 204 without having to send a request message.

Again, similar to the EMR interface 402, the example interface 406 is configured to transmit received data 206 to the node processor 404. In some embodiments, the interface 406 may queue the data 206 until the node processor 404 is available. Further, in some embodiments, the interface 406 may be configured to convert the patient data from a first format into a second format compatible that is for processing by the node processor 404 or storage in a graph database. For example, the interface 402 may be configured to convert patient data 206 from a text format to an ASCII format for storing data to an EMR or graph database. In some embodiments, the interface 406 converts the data 107 into a JSON, HTML, text, or nonSQL, format.

Before the outbreak management server 102 can create graph databases, a foundation or framework for the graph databases has to be defined. The example server 102 includes a graph configuration interface 408 and a graph configurer 410 configured to enable a user to configure conditions 412 for outbreak detection and conditions for determining possible/suspected, possible, and confirmed cases. The interface 408 and the configurer 410 are also configured to enable a user specify a hierarchy of node types in a location, such as a hospital. The user-provided or system-generated information 412 is stored to a database 414 as an outbreak graph template or definition file.

It should be approached that the conditions 412 do not specify or define the structure of an outbreak graph database itself, but rather the conditions for creating different node types, determining relationships between nodes, or determining values for writing to one or more node parameters. For example, the database 414 may store a different type of graph database template for each type of disease, infection or outbreak. The Official Journal of the European Union—Commission Implementing Decision of Aug. 8, 2012 laying down case definitions for reporting communicable diseases, dated Sep. 27, 2012, which is incorporated herein by reference, defines preconditions (if appropriate), clinical criteria, and diagnostic criteria for numerous diseases or infections. In addition, the document specifies epidemiological criteria and case classification criteria (i.e., a definition of a possible/suspected case of an infection, a definition of a probable case of the infection, and/or a definition of a confirmed case of the infection) for each infection or outbreak condition. The case classification criteria are based on the clinical criteria and the diagnostic criteria. Together, this information is used for specifying parameters and determining a case classification for a case definition of diseases or infections of a host node in a graph database. In addition, the epidemiological criteria specify contraction criteria, which are used for assessing relationships between hosts to determine possibilities or susceptibility to a host or other individual's contraction of an infection.

In an example regarding Q Fever (Coxiella burnetii), the clinical criteria is defined as any person with at least one of the following three symptoms: fever, pneumonia, or hepatitis. Laboratory criteria are defined as at least one of: isolation of Coxiella burnetii from a clinical specimen, detection of Coxiella burnetii in nucleic acid in a clinical specimen, or a Coxiella burnetii specific antibody response (IgG or IgM phase II). Epidemiological criteria include at least one of exposure to a common source or animal to human transmission. While there is no ‘possible’ classification case definition, a ‘probable’ classification case definition includes any person meeting the clinical criteria with an epidemiological link and a ‘confirmed’ case classification includes any person meeting the clinical and laboratory criteria.

In some embodiments, the graph configuration interface 408 is configured to receive the conditions 412 from a clinician or other user (from devices 114 or 126 of FIG. 1). The graph configuration interface 408 may, for example, provide an input user interface or fields prompting a user for the case conditions. The received conditions are processed by the graph configurer 410 to define the conditions for the specified outbreak. After the conditions are defined, the example node processor 404 is configured to determine if a patient's symptoms or other medical data 107 matches the specified conditions to determine if the patient has a ‘possible’, ‘probable’, or ‘confirmed’ classification case for the outbreak. If so, the node processor 404 creates an episode in the outbreak graph database 122 for the patient, which may include adding a graph or node for the patient to the existing graph database of other patients/nodes with the same disease or outbreak.

In other embodiments, the graph configuration interface 408 is configured to receive the conditions 412 as source information from, for example, an electronic version of “The Official Journal of the European Union”. In these embodiments, the graph configurer 410 is configured to read and parse the text to identify an infection name, preconditions, clinical criteria, diagnostic or laboratory criteria, epidemiological criteria, and case classifications. The graph configurer 410 automatically creates an outbreak template or definitions for each infection by populating the identified information into the appropriate parameters, nodes, relationships, etc. This configuration enables the interface 408 to be communicatively coupled to a health system or governmental database of infections. The interface 408 uses the connection to acquire new infection information as it is published, thereby increasing the speed at which an outbreak can be detected/tracked. It should be appreciated that outbreak conditions can also be determined for other types of outbreaks, such as food contamination or spoilage, chemical, radiation, etc.

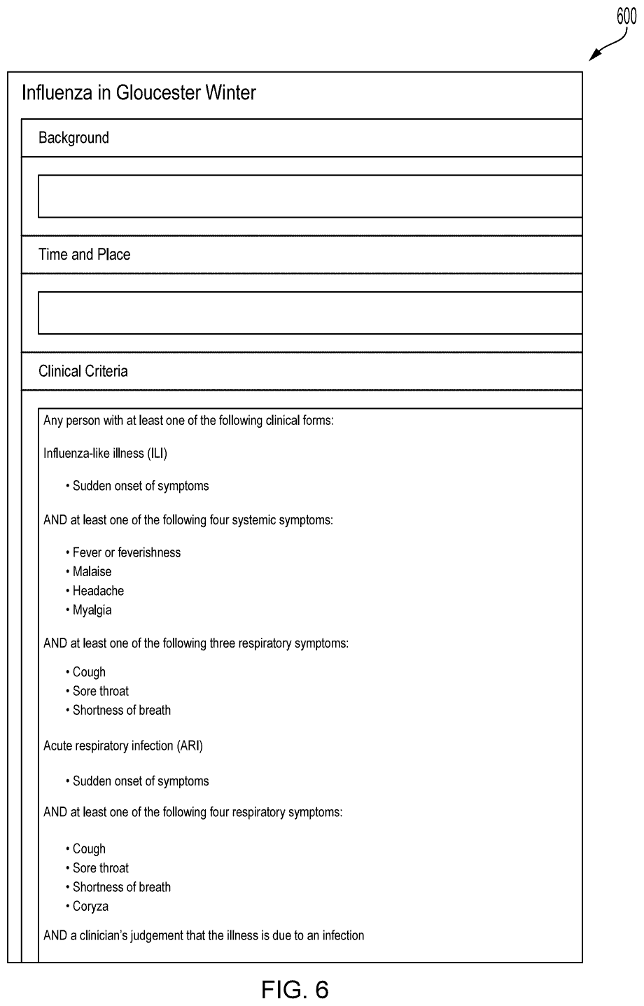

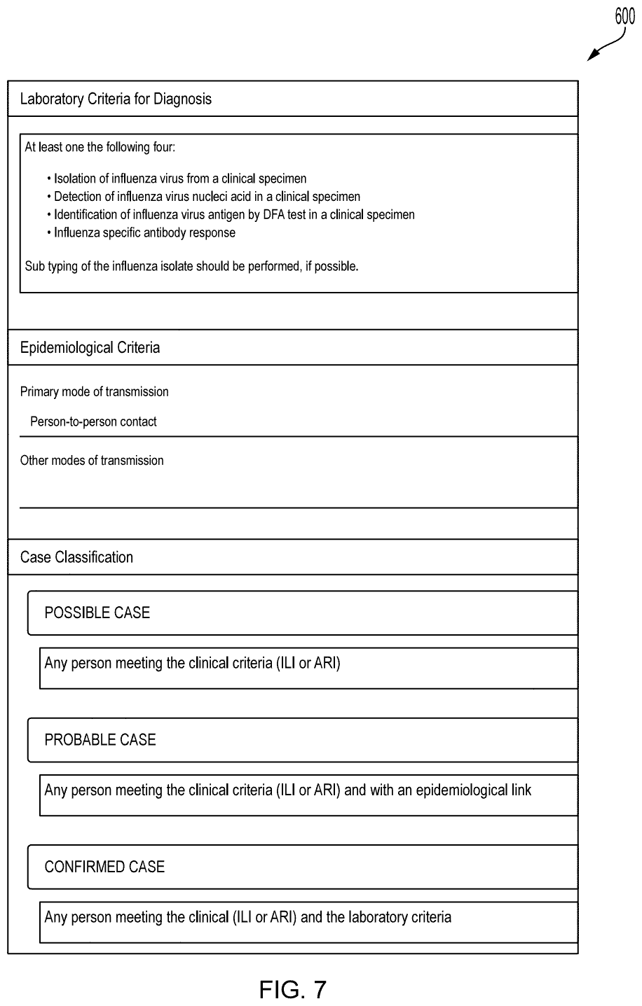

FIGS. 6 and 7 illustrate a graphical representation 600 of the conditions 412 of FIG. 4, according to an example embodiment of the present disclosure. The example graphical representation 600 of FIG. 6 includes clinical criteria for influenza. The example graphical representation 600 of FIG. 7 includes laboratory criteria for influenza, epidemiological criteria, and case classifications for ‘probable’, ‘possible’, and ‘confirmed’. The conditions 412 may be provided by a user or automatically extracted from a document or database. The example node configurer 410 is configured to create a definition file or template for an influenza outbreak graph database based on the information in the graphical representation 600. For example, the clinical criteria may be labeled as symptoms, with AND and OR logic being used to provide a computational relationship between the symptom in a definition file or template. In addition, the laboratory criteria may labeled as laboratory data types. Further, the node configurer 410 may use the epidemiological criteria to determine epidemiological links for the graph database that may correspond to higher weights for identifying potentially infected individuals. Further, the case classification includes Boolean logic of the laboratory and clinical criteria, which are used by the node configurer 410 for programming conditions for triggering ‘suspected’, ‘probable’, and ‘confirmed’ classifications in the graph database.

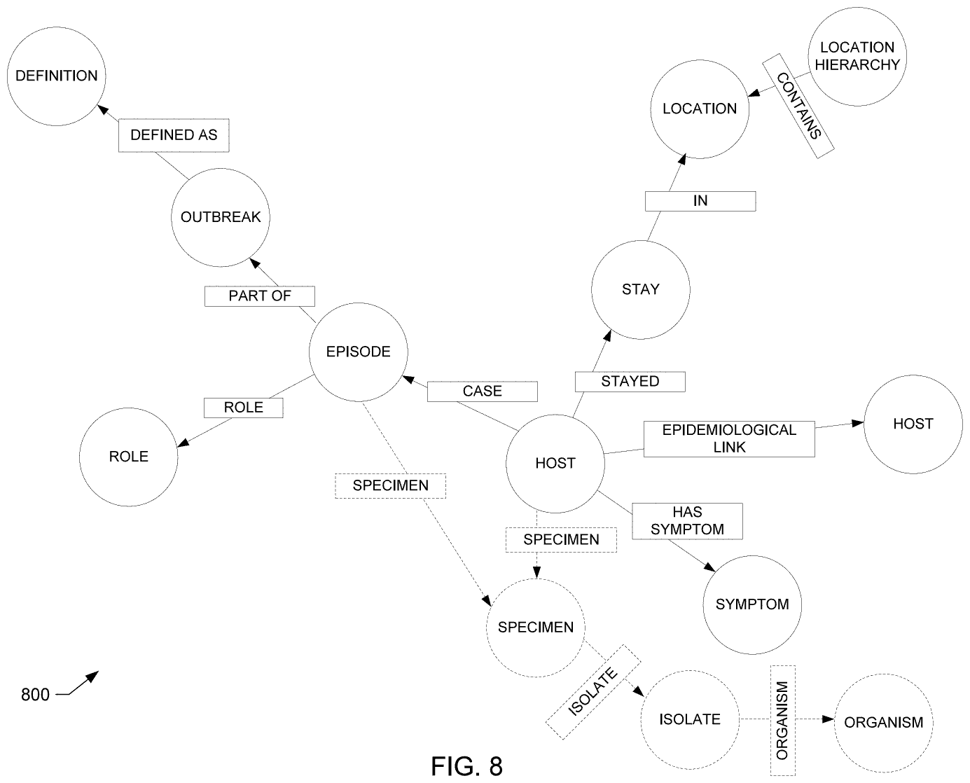

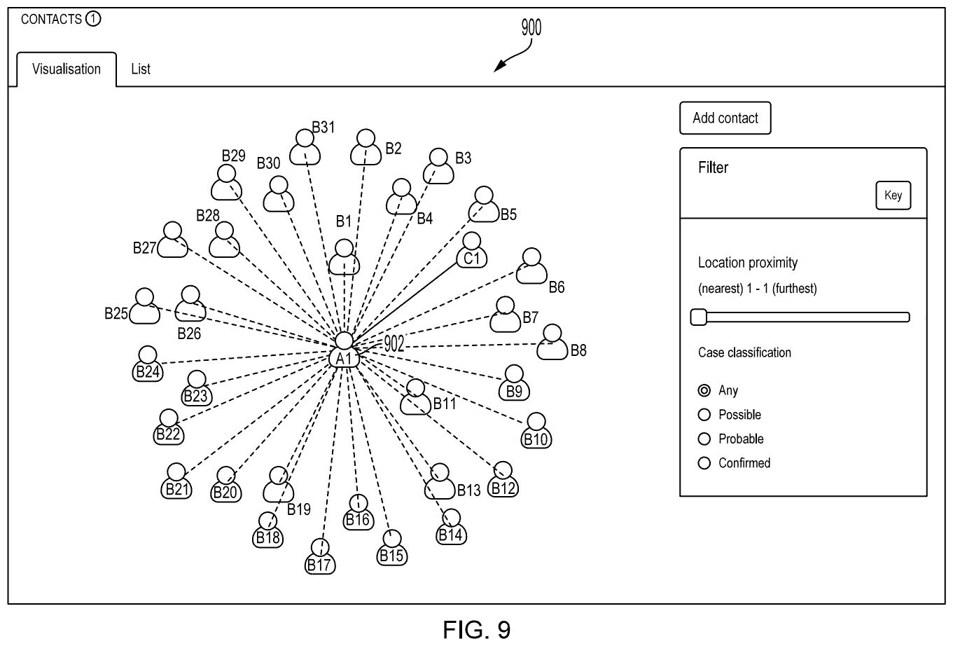

The example node processor 404 of FIG. 4 is configured to create outbreak graph databases for infections and other outbreak types that have been defined. The node processor 404 creates graph databases based on the graph database structure or framework 800 shown in FIG. 8, according to an example embodiment of the present disclosure. The example graph database structure 800 is specifically configured to record different data levels related to an outbreak. In other words, the graph database structure 800 defines relationships between different types of nodes (shown as circular elements) and the textural or sematic relationships (e.g., edges) between the nodes. The node processor 404 uses the graph database structure 800 for linking together nodes based on the specified format so as to only create meaningful links for downstream analysis. For example, the graph database structure 800 specifies that an Outbreak Node is defined by a Definition Node using a “DEFINED AS” edge or relationship. In addition, each occurrence of the outbreak is specified as an Episode Node which has a “PART OF” edge or relationship with the Outbreak Node. It should be appreciated that an unlimited number of Episode Nodes may be linked to an Outbreak Node representing hosts that are have classified cases of ‘confirmed’, probable′, or ‘possible’ with respect to the disease that is associated with the outbreak. The structure 800 prevents nodes other than Episode Nodes from being linked directly with an Outbreak Node, or even Outbreak Nodes from being linked to other Outbreak Nodes.

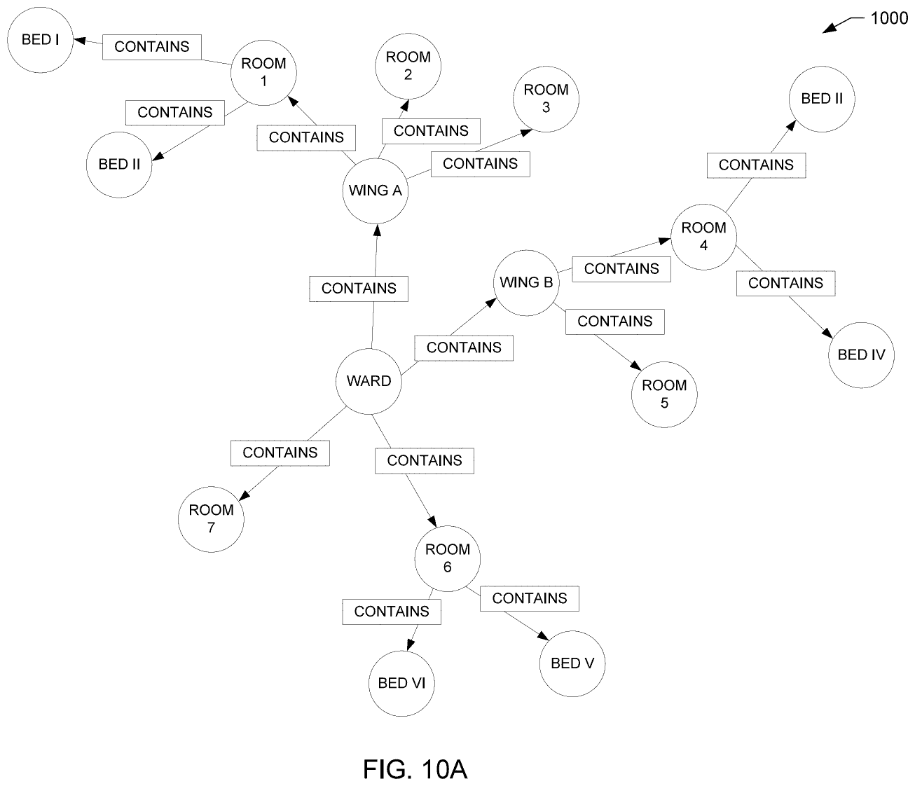

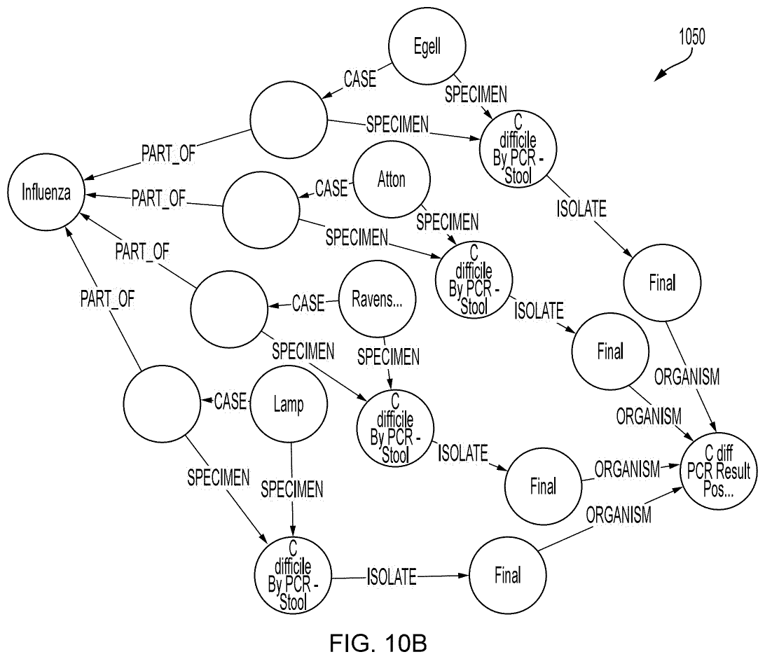

As illustrated in the structure 800 of FIG. 8, each Episode Node is connected to a Host Node via a “CASE” relationship or link. A Host Node may be connected to other Host Nodes via an epidemiological link, with at least some of the other Host Nodes having their own Episode Nodes linking back to the Outbreak Node (not shown). The Host Node is also connected to Symptom Nodes via a “HAS SYMPTOM” edge, relationship, or link. The Host Node may further be connected to one or more STAY Nodes via a “STAYED” relationship or link. The STAY Node is connected to a “Location Node” via an “IN” relationship or link. In some embodiments, the Location Node may be part of a larger hierarchy of Location nodes representative of a hierarchy of physical spaces in a location, as is shown in FIG. 10.

In the illustrated example, a Role Node is linked to the Episode Node via a “ROLE” edge, relationship, or link. This enables the graph database to reflect whether the same person or host acted as a carrier during an outbreak based on a certain role, such as clinician or patient. In some embodiments, an Outbreak Node may be linked to a Notes Node via a “HAS NOTE” relationship or link. The Nodes Node may specify information related to the outbreak.

The example node processor 404 is configured to generate an outbreak graph database when a patient has at least a suspected case of an infection or condition related to the outbreak. The node processor 404 may create separate outbreak graph databases for specific locations until there is sufficient data indicative of a spread of the outbreak to larger areas. For example, the node processor 404 may create different outbreak graphs for patients in different locations of a town. However, as the data provides new cases and interrelationships between patients, the node processor 404 may have sufficient information to combine the graphs together via epidemiological links to other host nodes.

The following tables provide parameters (e.g., attributes) for each of the different types of nodes shown in FIG. 8. Every Outbreak Node is connected to a Definition Node, which contains a case definition for the outbreak. As illustrated, there is a single Definition Node for each Outbreak Node. Table 1 below shows parameters of the Definition Node. Values for the parameters are identified or received from the conditions 412, shown in FIG. 4. It should be appreciated that the parameters or attributes shown in Table 1 are only illustrative and that the table may contain additional or fewer parameters or attributes. For example, some outbreaks may not have ‘suspected’ classification criteria.

TABLE 1

| Attribute | Description |
|---|---|
| name | Name (currently “Definition”) |
| background | Background to the case (HTML) |
| time_place | Information on the time and place of the outbreak/incident (HTML) |
| clinical criteria | Clinical criteria (HTML) |
| lab criteria | Laboratory criteria (HTML) |
| primary_mode_of | transmission Primary mode of transmission (relationship name) |
| other modes | of transmission Other modes of transmission (JSON object of multiple relationships) |
| suspected_enabled | Possible/suspected case classification applicable to this outbreak |
| | (boolean) |
| probable_enabled | Probable case classification applicable to this outbreak (boolean) |
| suspected | Criteria for determining a possible/suspected case (HTML) |
| probable | Criteria for determining a probable case (HTML) |
| confirmed | Criteria for determining a confirmed case (HTML) |
| notes | Additional notes (HTML) |
| created_at | When the node was created (populated by NeoEloquent) |
| updated_at | When the node was last updated (populated by NeoEloquent) |

For any given outbreak, there exists Hosts (e.g., infection sources) who have cases in Outbreaks. The cases are labeled as Episode Nodes. The following cypher query for the graph database illustrates an example host linkage to an outbreak:

People who have cases in an outbreak

MATCH (p:Host) - [:CASE] −> (e:Episode) - [:PART_OF] −> (o:Outbreak) RETURN p, e,
o

The cypher query may be filtered for non-null case classifications so as to focus on individuals who are ‘affected’ by the outbreak. When people (e.g., hosts) are added to an outbreak, the server 102 is configured to create the same relationship (i.e. with a connecting Episode Node), but a case classification may not be initially set. This is so that there is a short-list of people or hosts for investigation before ultimately assigning case status. This process is followed whenever a host is added to an outbreak or when individual hosts are synchronized from a connected server 204. Table 2 below shows parameters of an Episode Node. The node processor 404 determines the parameter values for the Episode Node from, for example, a patient's medical data 107. The start date/start is of the Episode Node is used to determine the ‘incidence’ (e.g. an outbreak epicurve) to indicate when the host or individual became part of the outbreak. The classification may change at any time as a host may transition in between ‘possible’, ‘probable’ & ‘confirmed’ case classifications. The node processor 404 uses the Definition Node to determine the case classification for the Episode Node. For example, symptoms and laboratory data 107 of the patient are matched by the node processor 404 to the case classification conditions in the Definition Node to determine if the patient is suspected, possible, or confirmed for a disease associated with the outbreak. In other examples, a clinician may provide or enter the classification. In these other examples, the node processor 404 may determine a recommended classification, which is displayed to a clinician to confirm after reviewing the patient's information in relation to the classification conditions.

TABLE 2

| Attribute | Description |
|---|---|
| start date | Human readable Y-m-d H:i:s date of the |
| | start of the case |
| start_ts | Timestamp version of the above |
| | (better for querying) |
| classification | NULL or ‘?’(possible), ‘P’ (probable), |
| | ‘C’ (confirmed) |
| immunization_status | Immunization status at case onset |
| | (j son terminology source) |
| immunization_type | Type of immunization (j son terminology source) |
| acquisition_of_infection | e.g. hospital-acquired (j son terminology source) |
| | If person died was the outbreak/incident |
| | organism/related |
| death certificate | condition recorded on the death certificate? |
| created_at | Automatically set by NeoEloquent |
| updated_at | Automatically set by NeoEloquent |

In any single outbreak, a single Episode Node may be identified as the index case. This is identified by a one-to-one relationship in between the Outbreak Node and the Episode Node called INDEX.

The example Host Node includes parameters that provide information related to the host. This includes a parameter providing an indication or flag as to whether the host is a person, animal, fomite, object, etc. The Host Node may also include parameters regarding a name, demographics, physical attributes, medical data 107, or other information related to a person. The node processor 404 may analyze or parse the data 107 and 206 for information to populate the parameters. In some examples, the node processor 404 may use word maps, natural language matches, label/field identifiers, and/or metadata to determine a property for population.

The Role Node shown in FIG. 8 may be linked to the Episode Node and provide a role for at least each some Outbreak Nodes that are linked to people. Table 3 below shows examples of roles, which include a patient, staff, and public. It should be appreciated that an Episode Node may have more than one Role Node, where for example, staff of a hospital may eventually also be a patient.

TABLE 3

| Role ‘role’ | Attribute | Description |
|---|---|---|
| Staff | stopped_work | Date staff member stopped work |
| Staff | returned_to_work | Date staff member returned to work |
| Patient | discharge_delayed | Whether or not discharge/transfer |
| | | was delayed as a result |
| | | of the outbreak/incident (Y/N) |
| Patient | surgery_cancelled | Where or not surgery was |
| | | cancelled as a result of the |
| | | outbreak/incident (YIN) |

The example Symptom Node is linked to a Host by the node generator 404 of the server 102 using, for example, the following cypher query:

Cypher query to see people and symptoms

MATCH (p: Person) - [:HAS_SYMPTOM] −> (s: Symptom) RETURN p, s

The node generator 404 may determine symptoms from the patient's medical data 107 by, for example, searching for symptom keywords. Table 4 below shows example parameters or attributes of the Symptom Node that may be determined by the node generator 404. The example node generator 404 uses the symptom parameters to determine a case classification of a host for the Episode Node using the defined criteria. As new symptoms are received, the host generator 404 adds the new symptoms as new Symptom Nodes that are connected to the Host Node, and updates a case classification accordingly. In addition, if a symptom ends, the host generator 404 notes the ending of the symptom. In some embodiments, the node generator 404, via user interface 420, receives an input message 422 from device 114 or 126. The input message 422 includes symptom information provided by a clinician. The message 422 may also include a case classification. In some embodiments, the user interface 420 may display an input window for a selected patient that enables a user to select a symptom from a drop-down list and/or select an icon representative of the case classification

TABLE 4

| Attribute | Details |
|---|---|
| uuid | Unique identifier |
| symptom | Symptom—see constants in App\Symptom |
| start_date | start date of symptom (ATOM format) |
| end_date | end date of symptom (ATOM format) |
| created_at | Automatically set by NeoEloquent |
| updated_at | Automatically set by NeoEloquent |

The example graph database structure 800 of FIG. 8 illustrates that Host Nodes are linked together via epidemiological links. Table 5 below shows examples of types of epidemiological links. In some examples, Table 5 may include a risk score or weight based on the link that corresponds to how a disease is typically transmitted. Links that are not associated with transmission maybe assigned a lower risk score when a database analyzer 430 of the server 102 searches for potential infected hosts. In other instances, Table 5 may contain only epidemiological links that are relevant towards transmission.

TABLE 5

| Attribute | Description |
|---|---|
| AIRBORNE | Airborne |
| ANIMAL_RESERVOIRS | Animal reservoirs |
| ANIMAL_TO_PERSON_CONTACT | Animal to person contact |
| CONTAMINATED_OBJECTS | Contaminated objects |
| DROPLET_SPREAD | Droplet spread |
| ENVIRONMENTAL_RESERVOIRS | Environmental reservoirs |
| FOOD_AND_DRINKING_WATER | Food and drinking water |
| INSECT_BITES | Insect bites |
| PERSON_TO_PERSON | |
| CONTACT | Person-to-person contact |

In some embodiments, the node generator 404 is configured to determine epidemiological links (or potential links for confirmation) between hosts using data 107 and/or 206. For example, social media or demographic relationship data may be used to determine epidemiological links. In other examples, geographic location may be used to determine when two hosts were in the same location at the same time. In other examples, a clinician's notes may include a list of individuals that came in contact with a patient. In yet other examples, a treatment schedule of a patient, included within the data 107, may identify which clinicians came in contact with a patient. The node generator 404 is configured to use word maps, fields, labels, or metadata to identify names from the data 107 and/or 206 for identifying hosts and potential epidemiological links between the hosts.

In some embodiments, the user interface 420 of FIG. 4 may display an interface screen 900 that provides a graphical representation of links or potential links between hosts, for a given selected host 902. Upon request, the database analyzer 430 analyzes links (or links labeled as ‘potential links’) between a selected host and other hosts to determine and render the graphical representation. A clinician interacts with the interface screen 900 by selecting potential links and assigning a corresponding epidemiological link. Selection of the epidemiological link by the clinician causes the node generator 404 to update a link between selected hosts in the graph database with the received epidemiological link. For example, a potential link is changed by the node generator 404 to an epidemiological link.

Returning to FIG. 8, the Host Node is linked to a Location Node via a Stay Node. The Stay Node specifies start and end dates and/or times in which a host was at a particular location. Table 6 below shows example parameters or attributes of the Stay Node. The example node generator 404 determines the date/time, for example, from the patient medical data 107 corresponding to patient admittance and tracking within a medical facility. For example, a patient record may indicate that a patient was located in Bed A of Room 1774 of an Acute Care Ward or care area for a week in April 2018. The node generator 404 uses metadata, data labels, fields, etc. to locate the date, which it uses to populate as a value into the appropriate parameters or attributes of the Stay Node. In other examples, a user may provide the dates/times via the user interface 420. Further, the node generator 402 may use geolocation data 206 to determine date/time information. The example date/time information may be used by the data analyzer 430 to determine which hosts were in the same location at the same time.

TABLE 6

| Attribute | Description |
|---|---|
| start_date | Human readable start date of the stay |
| start_ts | Timestamp start date of the stay |
| end_date | (Optional) human readable end date of the stay |
| end_ts | (Optional) timestamp end date of the stay |
| readonly | Node may not be edited in the UI (for stays |
| | imported from a third-party system) |
| created_at | When the node was created—Recorded |
| | automatically by NeoEloquent |
| updated_at | When the node was last updated—Recorded |
| | automatically by NeoEloquent |

The Location Node includes parameters that identify a location. Table 7 below shows an example of parameters for a Location Node. In addition, a location node may include parameters for GPS coordinates, a street address, a building name, and/or a place of interest. The node generator 404 uses the data 107 and/or 206 to determine values for the parameters or attributes similar to determining a date/time for the Location Node.

TABLE 7

| Attribute | Description |
|---|---|
| uuid | Unique identifier of a location |
| name | Name of the location |
| type | Type of the location: e.g., room, bay, hospital group, etc. |
| risk | Location proximity risk score (see above) |
| readonly | Set to true if the location data is imported from a |
| | third party system |
| | (see below′) |
| created_at | When the node was created—Recorded automatically by |
| | NeoEloquent |
| updated_at | When the node was last updated—Recorded automatically |
| | by NeoEloquent |

In many embodiments, a location is part of a larger hierarchical location. In these embodiments, the link to the Stay Node corresponds to a Location Node lowest in the hierarchy. The hierarchy may correspond to an organization in a geographic location or physical space, such as a hospital. The example graph configurer 410 of FIG. 4 is configured to automatically create a hierarchical location map 1000, as shown in FIG. 10A from imported data, which may specify a hierarchical structure of a location. In other examples, the graph configurer 410 enables a user to create the hierarchical location map 1000. As shown in FIG. 10A, each sub-location is linked to a higher-order location via a “CONTAINS” relationship or link. In the illustrated example, a hospital Ward Location Node contains Wing Location Nodes A and B, which each contains a number of Room Location Nodes. In addition, some Room Location Nodes directly connected to the Ward Location Node without connection to a Wing Node. Further, some Room Location Nodes connect to separate Bed Location Nodes. While the map 1000 in FIG. 10A corresponds to a hospital, in other examples, the graph configurer 410 may create relational maps for businesses, neighborhoods, cities, commercial/residential buildings, public spaces, transportation systems, etc. The Location Nodes are used by the database analyzer 430 to determine a relational and/or physical distance between hosts during designated times/dates.

In some embodiments, the Location Nodes may include a risk score, which provides a numerical indication of a risk of infection of hosts corresponding to a distance between locations. The example database analyzer 430 may use the risk scores for locations to identify potential hosts that have epidemiological links to other hosts. For example, patients in adjacent beds may be assigned relatively high risk scores for their respective Bed Nodes. Table 8 below shows an example of risk scores for different Location Nodes.

TABLE 8

| Node | | Risk |
|---|---|---|
| Type | Description | Score |
| Location: Bed | A space that is allocated for sleeping/laying on. This is not the | 1 |
| | physical bed/trolley that may be moved about, but the space it may | |
| | occupy | |
| Location: Room | A space that is allocated as a room | 2 |
| Location: Corridor | Any corridor within a Building that may connect room | 3 |
| Location: Ward | A Ward is a section of a medical facility that may contain rooms and | 4 |
| | other types of location | |
| Location: Level | A Level in a multi-level Building/Structure | 5 |
| Location: Wing | A Wing within a Building, this often contains levels, rooms and | 6 |
| | corridors | |
| Location: Structure | Any non-residential building or structure. This may contain rooms, | 7 |
| | corridors, wings, etc. It may not have walls or a roof, but is | |
| | considered a defined/allocated space | |
| Location: Building | A dwelling including private houses, flats, hospitals, and hotels. | 8 |
| | Usually used to reference a location where a person/patient may | |
| | reside | |
| Location: Street | A defined path to travel between 2 points that has a known name | 9 |
| Location: Site | A collection of buildings or other locations such as a site or a | 10 |
| | campus | |
| Location: Hospital | A medical facility specific collection of buildings | 10 |
| Location: Area | A zone, region, postcode, zip code | 11 |
| Location: HospitalGroup | A group of hospitals such as a Health Board/Authority | 11 |
| Location: County | A county, province, or state | 12 |
| Location: Country | A nation with its own government | 13 |

In some embodiments, the Ward Location (or other centralized Location Node) includes parameters or attributes that provide general (e.g., survey) information relevant for the location. The parameters may be related to the outbreak and/or related to an activity level of the location. In some embodiments, the survey information may be its own node (e.g., a Survey Node), which is linked to the Location. Table 9 shows an example of parameters or attributes of a Survey or Location Node regarding activity level. The user interface 420 enables a user to provide the information in Table 9. Additionally or alternatively, the node generator 404 is configured to read hospital patient tracking information to determine an overall activity for a particular location. For example, the node generator 404 may determine a number of patients in beds for a care area compared to a total number of beds. The database analyzer 430 may display the information in Table 9 to show how activity level of a location changes or corresponds to an outbreak.

TABLE 9

| | |
|---|---|
| Attribute | Description |
| date | Human readable date (Y-m-d) |
| date_ts | Timestamp of the date of the survey (at 00:00 hours) |
| specialty | Specialty of the ward (text) |
| closed | Boolean: whether or not the ward is closed |
| rooms | Number of bays/rooms |
| rooms_closed | Number of bays/rooms closed |
| cohorted | Number of cohorted patients |
| empty_beds | Number of empty beds (at 10am) |
| isolation_rooms | Number of isolation rooms |
| staffed_beds | Number of staffed beds |
| created_at | When the node was created—recorded automatically by NeoEloquent |
| updated_at | When the node was last updated—recorded automatically by NeoEloquent |

In some embodiments, the example node processor 404 receives batch information regarding patients in a hospital during a time period related to an outbreak. In these embodiments, the node processor 404 may create clusters of nodes that are linked together by the hospital Location Nodes. As more property values are identified from received patient medical data 107, the node processor 404 determines relationships (e.g., epidemiological links) between the patients. Table 10 below shows nodes and relationships of a graph database that the node processor 404 may use to synchronize different clusters of one or more patients among the same location. The database analyzer 430 determines, for example, from the data in Table 10 that an outbreak is local to a particular hospital ward or care area or room level.

TABLE 10

| Node | Details |
|---|---|
| Host: Person | Each patient will have a unique Host Person node |
| Stay | Each period of ward/room/bed occupancy will be represented by a Stay |
| | node |
| Location: | A hospital group location |
| HospitalGroup | |
| Location: Hospital | A hospital location |
| Location: Ward | A ward location |
| Location: Room | A room level location |
| Location: Bed | A bed level location |

In some instances, the node processor 404 is configured to create a single graph database for an outbreak type per location. As new patients are admitted or include symptoms, the example node processor 404 is configured to add the patients as Host Nodes to the graph database. Tracking patients that do not show symptoms, but are in the same location as an outbreak, enables the database analyzer 430 to identify patients that are vulnerable to an outbreak.

In some embodiments, the node processor 404 is configured to include microbiology information within a graph database. As illustrated in FIG. 8, each Host Node may be connected to a Specimen Node via a “SPECIMEN” edge, relationship, or link. Additionally, the corresponding Episode Node of the Host Node may also have a “SPECIMEN” edge or relationship with the Specimen Node. This linking accounts for a Host potentially having many different microbiology specimens, which may or may not be relevant to an outbreak. Table 11 shows example parameters or attributes of the Specimen Node.

TABLE 11

| Attribute | Description |
|---|---|
| uuid | string “{lab_request_id}#{specimen_number}# |
| lab_request_id | The laboratory request ID |
| specimen_number | The specimen number |
| readonly | (boolean) indication that this is imported data so is read only in the UI |
| status | The PFA status of the specimen result (Preliminary, Final or Amended) |
| taken | Human readable date/time of when the specimen was taken (Y-m-d H:i:s) |
| taken_ts | Timestamp when the specimen was taken |
| lab | Laboratory where the specimen was taken |
| type | Specimen type |
| created_at | When the node was created |
| updated_at | When the node was last updated |

Each Specimen Node may be linked to an Isolate Node via an “ISOLATE” edge or relationship. The Isolate Node specifies an isolate within specimen results. Table 12 below shows example parameters for the Isolate Node.

TABLE 12

| Attribute | Description |
|---|---|
| uuid | String “{lab_request_id)#{specimen_number}# |
| | {organism_code}” |
| readonly | (boolean) indication that this is imported data |
| | so is read only in the UI |
| result_status | PFA result status of the isolate |
| | (Preliminary, Final, or Amended) |
| create_at | When the node was created |
| updated_at | When the node was last updated |

An Isolate Node may be connected to an Organism Node via an “ORGANISM” edge or relationship. The Organism Node identifies a single organism found in an isolation routine. Table 13 below shows example parameters of the Organism Node.

TABLE 13

| Attribute | Description |
|---|---|
| uuid | Code for the organism (the organism code from ICNet) |
| readonly | (boolean) indication that this is imported |
| | data so is read only in the UI |
| name | Name of the organism |
| created_at | When the node was created |
| updated_at | When the node was last updated |

In some examples, the node generator 404 may be configured to use the Preliminary, Final, Amended, and Deleted (“PFA(D)”) system for the specimen result and isolate level. FIG. 10B illustrates a graph database 1050 that includes Specimen Nodes, Isolate Nodes, and Organism Nodes, according to an example embodiment of the present disclosure. The graph database 1050 shows that specimens and isolates from different hosts are traced back to the same organism. Such information is useful by not only showing a spread of an outbreak, but also a biologic that is responsible (or suspected) for the outbreak.

In some examples, the graph database may replace the specimen, isolate, and organism with similar nodes for identifying exposure to chemical substances, radiation types, etc.

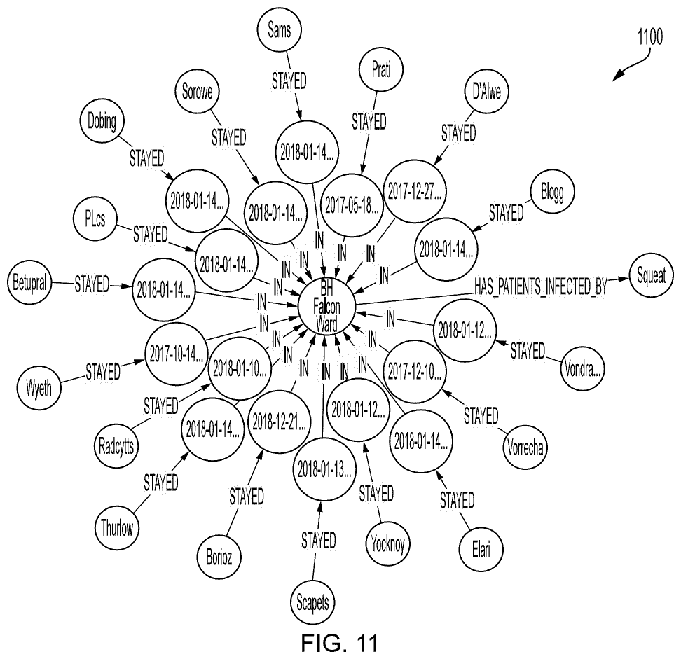

FIG. 11 illustrates an example graph database 1100 for a Ward Location Node, according to an example embodiment of the present disclosure. The graph database 1100 may be rendered by the database analyzer 430 in response to view relationships relative to the Ward Location Node. In this example, the database analyzer 430 may compress or hide the location hierarchy to view Stay and Host Nodes that have a relationship with the Ward Location Node. In some instances, the database analyzer 430 is configured to color code the Host Nodes based on their case classification to an outbreak to visually indicate the spread of an outbreak relative to the Ward Location Node.

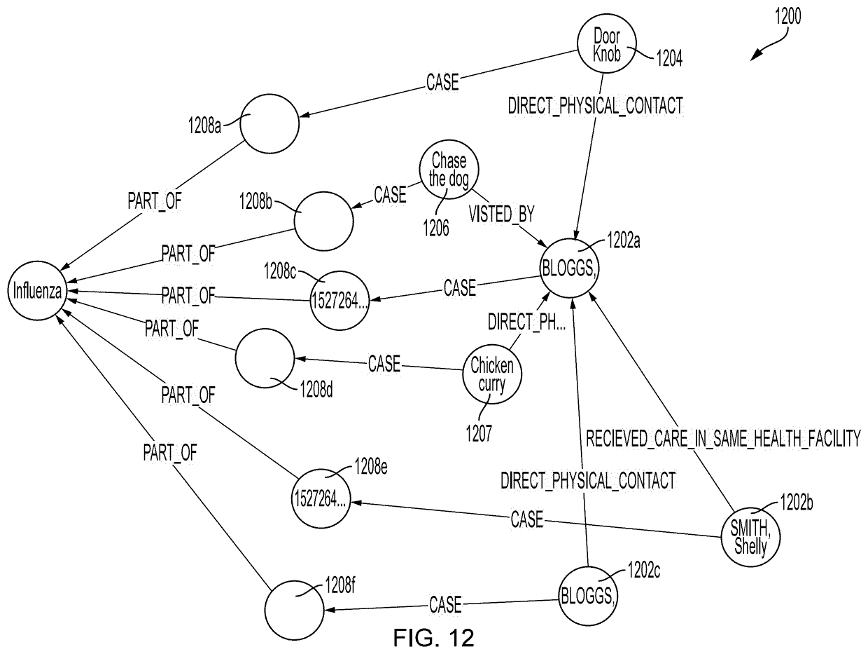

FIG. 12 illustrates an example graph database 1200 for an Influenza Outbreak Node, according to an example embodiment of the present disclosure. In this example, different types of host nodes are shown, with Host Nodes 1202 corresponding to persons and Host Nodes 1204, 1206, and 1207 corresponding to fomites or objects. The example node generator 404 is configured to generate a separate Episode Node 1208 for each of the Host Nodes. However, only the Episode Nodes 1208c and 1208e that are linked to a confirmed or probably case of influenza in a person are provided a case number. In contrast, the other Episode Nodes 1208a, 1208b, 1208d, and 1208f are not linked to a confirmed or probably case of influenza and accordingly are not assigned a case number. In addition, the example node generator 404 provides epidemiological links between the Host Nodes 1202, 1204, 1206, and 1208. In some instances, the node generator 404 determines the links from data 107 and 206. In other instances, the links are specified by a clinician via the user interface 420.

In some embodiments, different types of outbreaks may be part of the same graph database. In other words, each outbreak may be a separate cluster, with clusters linked together based on location and/or hosts. For example, for a particular, location, some hosts may be linked to a first outbreak while other hosts are linked to a second outbreak. The interrelation between outbreaks enables the database analyzer 430 to determine and display information regarding vulnerability of patients to certain overlapping outbreaks or determine correlation between different outbreaks. The database analyzer 430 is configured to filter a graph database for a single outbreak type by removing or hiding nodes that are related to other outbreaks.

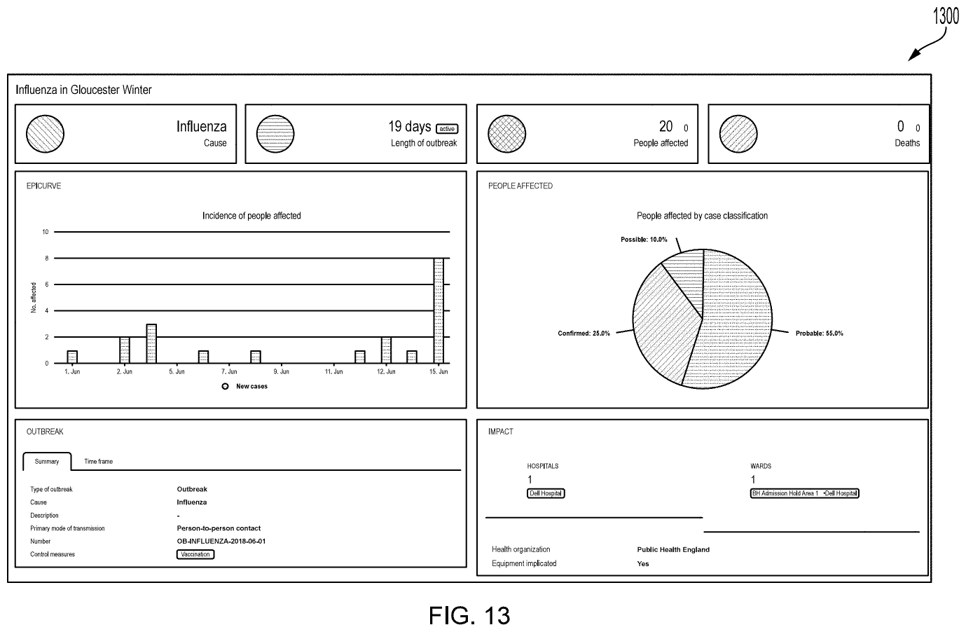

FIG. 13 shows a dashboard interface 1300 that is configured to be rendered by the database analyzer 430, according to an example embodiment of the present disclosure. The dashboard interface 1300 includes separate sections that provide an overview for a selected outbreak. The database analyzer 430 analyzes, for example, nodes related to the specified outbreak (e.g., influenza) to determine, for example, a length of the outbreak, a total number of people affected, a total number of deaths attributable to the outbreak, a timeline of incidence, and a graph of case classification.

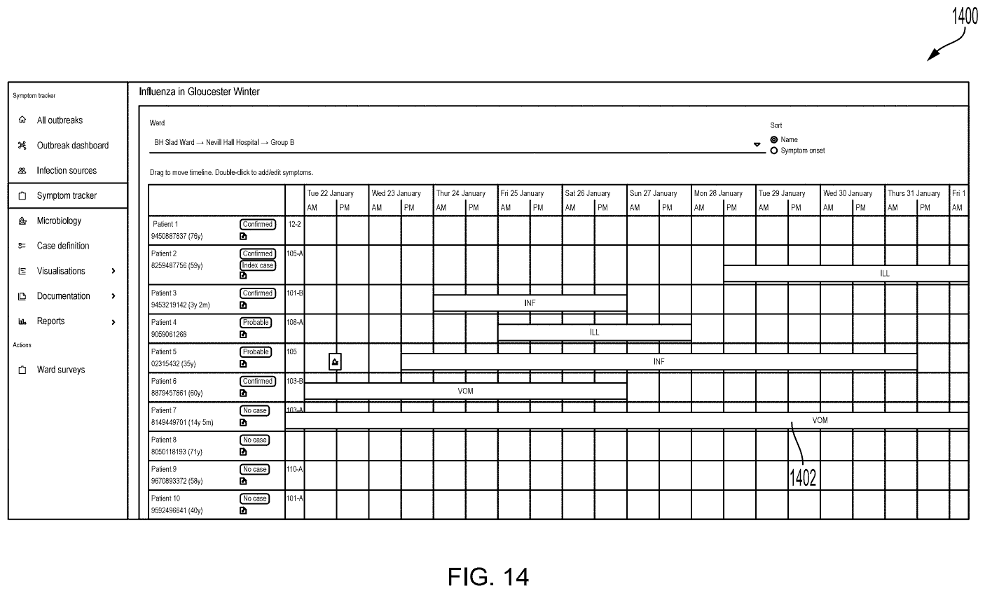

FIG. 14 shows a symptom tracker interface 1400 that is configured to be rendered by the database analyzer 430, according to an example embodiment of the present disclosure. The database analyzer 430 is configured to determine, using Host and Symptom Nodes, which patients had a particular symptom and the corresponding time period for the symptom. The interface 1400 also includes, under a patient name, a location of the patient or identifier and demographic information. Such information provides an indication of all patients showing at least one symptom that may be related to an outbreak. In some embodiments, the interface 1400 is interactive. For example, the interface 1400 may include a filter feature to filter by location, case classification, etc. In response to a request for filtering, the database analyzer 430 determines the appropriate information to display from the outbreak graph database. In other embodiments, the interface 1400 is configured to enable a clinician to move, expand, or shorten a time period for a symptom, such as symptom 1402 for ‘vomiting’. The interface 1400 transmits commends provided by the clinician to the node processor 404, which adjusts the parameters in the Symptom Node accordingly (e.g., changes the duration of the symptom).

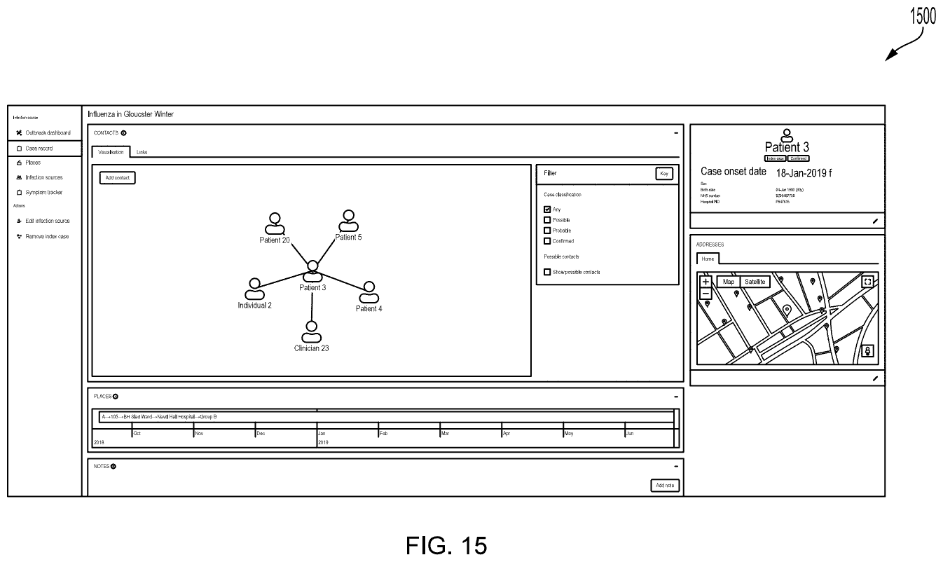

FIG. 15 illustrates a diagram of a patient interface screen 1500 for a patient (i.e., Patient 3) associated with a Host Node of an outbreak, according to an example embodiment of the present disclosure. The database analyzer 430 determines information to display in the interface screen 1500 based on patient information, relationship with other Hosts and Time/Date-Location information. A clinician may view the screen 1500 by selecting a patient in the screens/interfaces 1200 and 1400 or by providing a query entry of a patient's name or identifier. The interface 1500 may provide an overview of a patient's locations over time, including a map of those locations. The interface screen 1500 may also display a graphical relationship between the patient and other Hosts that the patient came into contact with at certain identified locations. The interface screen 1500 further shows which of those other Host Nodes have a confirmed or probable case of influenza, for example.

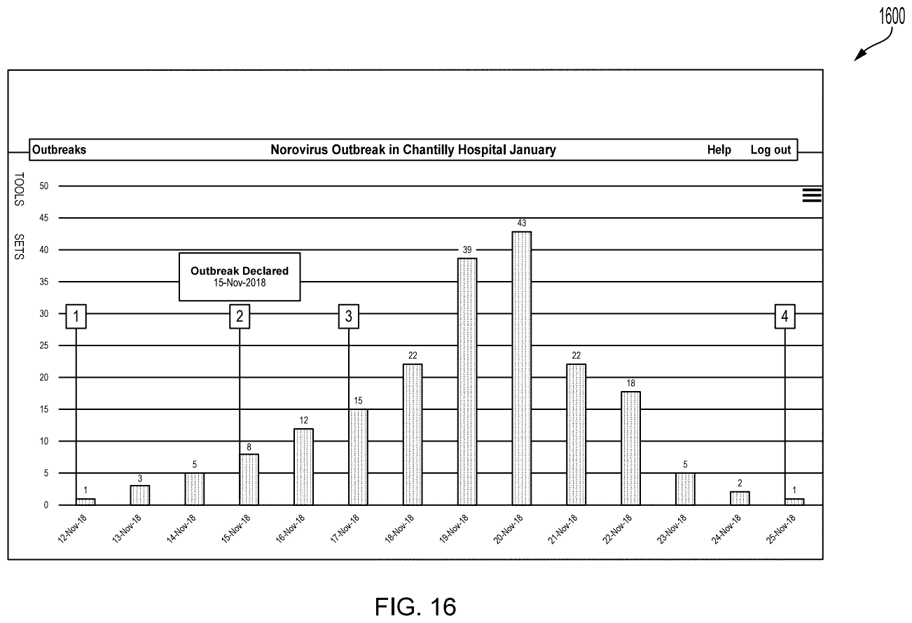

FIG. 16 illustrates a diagram of an outbreak interface screen 1600 for a Norovirus Outbreak, determined by the database analyzer 430, according to an example embodiment of the present disclosure. In the illustrated example, the database analyzer 430 receives a request to view dates of occurrence of the Norovirus at a specified location. In response, the database analyzer 430 analyzes a graph database for nodes related to Norovirus and compiles the information from case classifications of Hosts corresponding to patients/persons. In some examples, the interface screen 1600 may include options for selection that cause the database analyzer 430 to display confirmed cases compared to suspected and probable cases.

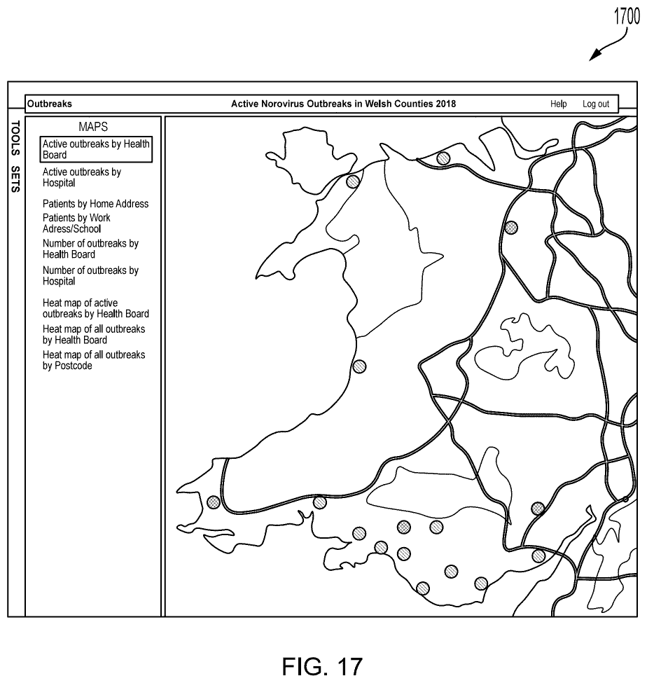

FIG. 17 illustrates a diagram of a map screen 1700 showing an outbreak of Norovirus for Welsh Counties, according to an example embodiment of the present disclosure. In the illustrated example, the database analyzer 430 receives a request to view a map of outbreaks of Norovirus for a specified location. In response, the database analyzer 430 analyzes a graph database for nodes related to Norovirus and the specified location. If a number of confirmed and/or probable cases exceeds a threshold for a particular location, the database analyzer 430 determines that an icon is to be placed on a map to indicate the presence of an outbreak in that location. In some embodiments, the threshold may be as low as one case. In some instances, the database analyzer 430 may select a color and/or size of an icon based on confirmed and/or probably cases for a particular area. A clinician may use tools with the map screen 1700 to zoom into a particular location to view, for example, individual cases by address and/or area in a medical facility. As a clinician changes a resolution of the map screen 1700, the database analyzer 430 may provide the icons at a higher resolution, such as by neighborhood, street, or residence rather than by town. To do this, the database analyzer 430 determines a scale of the displayed map, regions (e.g., neighborhoods, streets, addresses, etc.) within the display map, and confirmed and/or probable cases that correspond to the identified regions. The database analyzer 430 may also analyzer the outbreak information over time to provide an indication as to whether incidence is increasing or decreasing for a location and compare dates of incidence to determine a spread location.

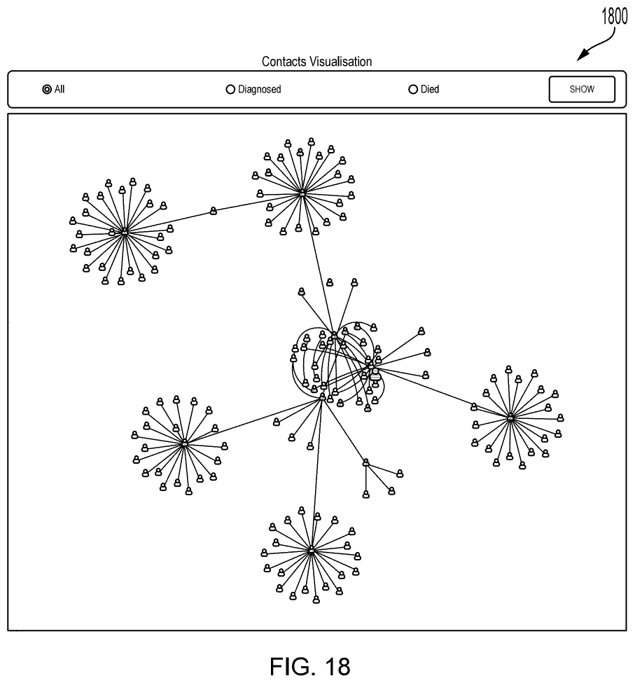

FIG. 18 illustrates a diagram of a contacts screen 1800 for an outbreak, according to an example embodiment of the present disclosure. In the illustrated example, the database analyzer 430 receives a request to view links between person hosts or nodes. The database analyzer 430 may identify confirmed, probable, or possible cases compared to all contacts for a given host. Such information may be used to quarantine or take preventive measures for certain individuals connected to an infected host. The screen 1800 may also show the interrelations among different hosts and how an outbreak is spreading.

III. Example Procedures for Outbreak Tracking and Management

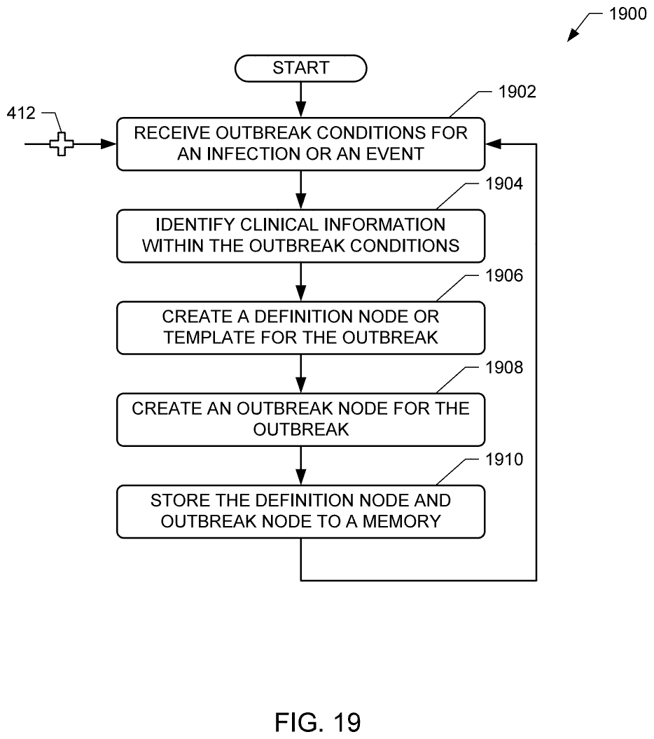

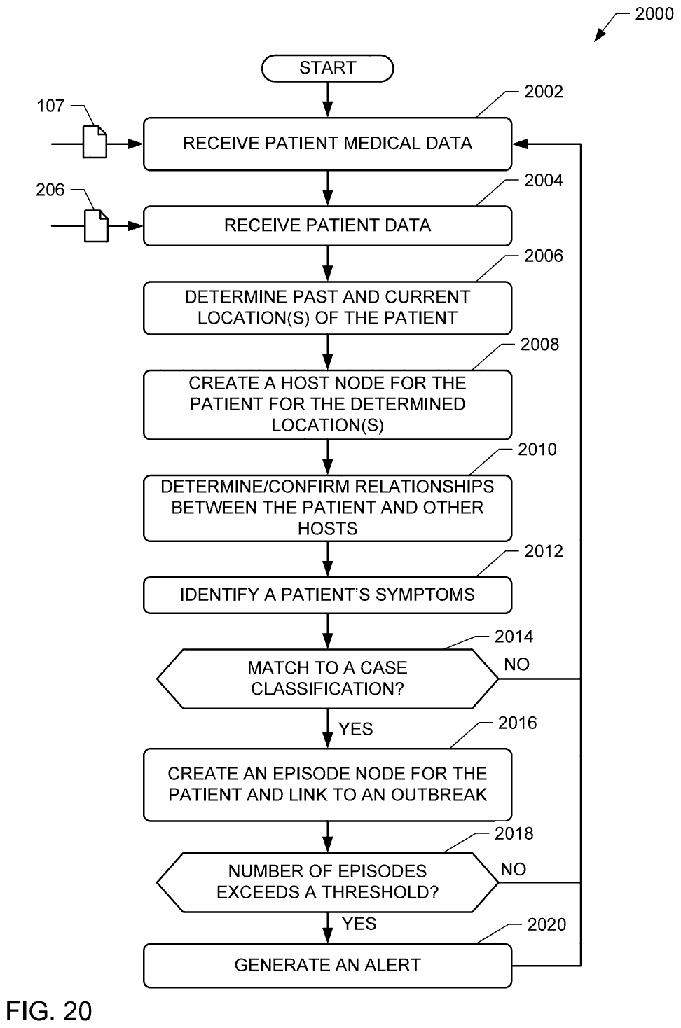

FIGS. 19 and 20 illustrate flow diagrams showing example procedures 1900 and 2000 to configure and create graphs databases, according example embodiments of the present disclosure. Although the procedures 1900 and 2000 are described with reference to the flow diagrams illustrated in FIGS. 19 and 20, it should be appreciated that many other methods of performing the steps associated with the procedures 1900 and 2000 may be used. For example, the order of many of the blocks may be changed, certain blocks may be combined with other blocks, and many of the blocks described are optional. Further, the actions described in procedures 1900 and 2000 may be performed among multiple devices including, for example the outbreak management server 102, the user device 126, the clinician device 114, the HIS 104, and/or the servers 204 of FIGS. 1 to 3. Further, the procedures 1900 and 2000 may be illustrative of the instructions 128 of FIG. 4.

The example procedure 1900 of FIG. 19 begins when the outbreak management server 102 receives one or more conditions 412 for an infection or event (block 1902). The conditions 412 may be received in a document, from a website, and/or entered by a clinician. The server 102 identifies clinical information within the outbreak conditions and creates a definition node, template, and/or file for the specified outbreak (blocks 1904 and 1906). As described above in regard to FIG. 4, this includes identifying case criteria and populating the criteria into parameters or attributes of a Definition Node for the outbreak. The server 102 may also create an outbreak node that is linked to the definition node (block 1908). The server 102 stores the definition node and the outbreak node to a memory (block 1910). The case criteria for the outbreak may then be used to determine if a host has an episode within the new outbreak. If so, the outbreak may be created in a new graph database or added to a current graph database for a corresponding location.

The example procedure 2000 of FIG. 20 begins when the outbreak management server 102 receives patient medical data 107 from the HIS 104 (block 2002). The outbreak management server 102 may also receive patient data 206 from the servers 204 (block 2004). The outbreak management server 102 then determines past and current locations of the patient (or person) based on the data 107 and/or 206 (block 2006). The outbreak management server 102 creates a Host Node and Location Nodes (and Stay Nodes) for the patient/person (block 2008). The outbreak management server 102 also determines relationships between the patient and other hosts (block 2010). The determined relationships are added to the graph database between the appropriate nodes. In some examples, the outbreak management server 102 may prompt a clinician to provide the relationships with other hosts or provide a confirmation of a potential determined relationship.

The example outbreak management server 102 also identifies a patient's symptoms from the data 107 and/or 206 (block 2012). From the identified symptoms, the outbreak management server 102 determines if the patient's symptoms match a case classification for one or more outbreaks (block 2014). If there is a match to an outbreak, the outbreak management server 102 creates an Episode Node and creates a link between the patient and the outbreak (block 2016). If there is not a match to a case classification, the outbreak management server 102 returns to block 2002 and receives data for the same patient or additional patients.

If the outbreak management server 102 creates an Episode Node, the server 102 may then compare the number of patient episodes (with the same or similar Episode Nodes) to a threshold to determine if an alert should be generated or the outbreak should otherwise be promoted for further attention (block 2018). If the threshold is exceeded, the management server 102 is configured to generate an alert or otherwise transmit a message or provide an indication that the outbreak should receive attention (block 2020). This may include, for example, the server 102 transmitting one or more text messages or push notifications to clinician devices 114. If the threshold is not exceeded and/or after an alert is generated, the outbreak management server 102 returns to block 2002 for processing newly received data for the same patient or other patients.

IV. Case Sift and Cluster Sift Embodiment

As discussed above, the outbreak management server 102 may start with an outbreak and identify patients that are part of the outbreak. Alternatively, the outbreak management server 102 may create an outbreak for each patient, then group the outbreaks together based on shared connections. As discussed below, in some alternative embodiments, the outbreak management server 102 may create a graph database structure at the disease level and use one or more rules (e.g., sift tools) to determine if the cases constitute an outbreak. The example rules are user configurable and configured to compare data in a graph database structure to determine if one or more cases or clusters meet the conditions for a designation of an outbreak. In some instances, the one or more rules may also specify a workflow that defines how the outbreak case or cluster is to be further processed, how the outbreak case or cluster is to be assigned, and/or steps/stages for managing the outbreak case or cluster.

As described herein, a case corresponds to an individual incident of a patient having a particular disease. A case may be classified as being suspected, probable, or confirmed for a particular disease and is represented by an Episode node in the graph database structure. A cluster refers to two or more cases within a predefined geographic area. Clusters are represented by outbreak nodes in the graph database structure, described above. The example outbreak management server 102 uses the one or more rules defined herein to determine when one or more cases constitute a disease cluster for designating an outbreak.

In some embodiments, the one or more rules provides for assigning certain cases of a cluster to one or more users (e.g., clinicians) for disease management. For example, a first user may manage a cluster while a second user is assigned ten cases in the cluster and a third user is assigned ten other cases in the cluster. The assignment may be made based on a user's responsibilities, working balancing rules, and/or a user's treatment location.

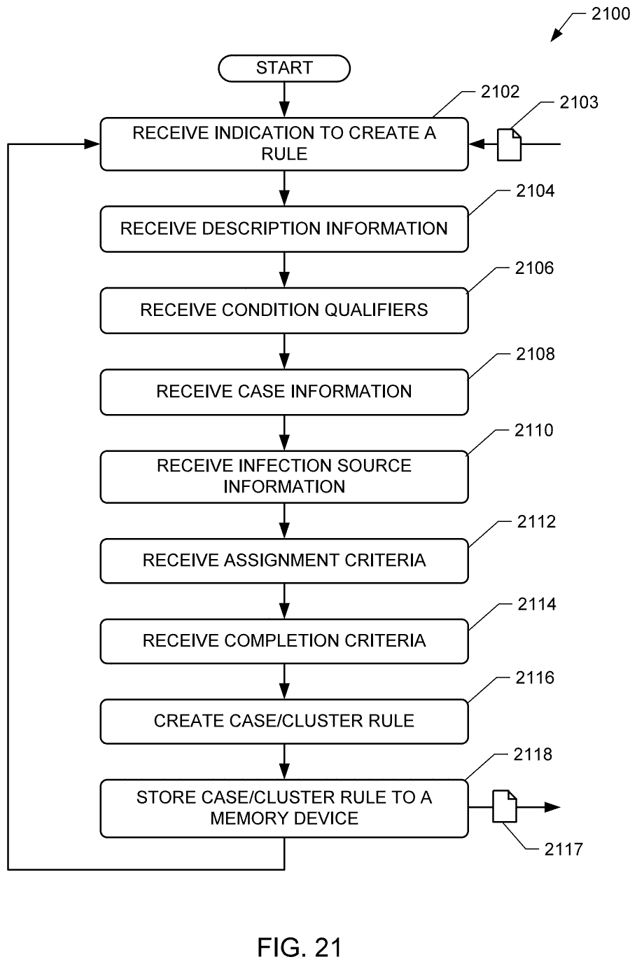

FIG. 21 illustrates a flow diagram showing an example procedure 2100 for creating one or more rules for case and/or cluster sifting for outbreak classification, according to an example embodiment of the present disclosure. Although the procedure 2100 is described with reference to the flow diagrams illustrated in FIG. 21, it should be appreciated that many other methods of performing the steps associated with the procedure 2100 may be used. For example, the order of many of the blocks may be changed, certain blocks may be combined with other blocks, and many of the blocks described are optional. Further, the actions described in the procedure 2100 may be performed among multiple devices including, for example, the outbreak management server 102, the user device 126, the clinician device 114, the HIS 104, and/or the servers 204 of FIGS. 1 to 3. Further, the procedure 2100 may be illustrative of the instructions 128 of FIG. 4. While reference is made to creating rules for cases, the same procedure 2100 may be used for creating rules for clusters.

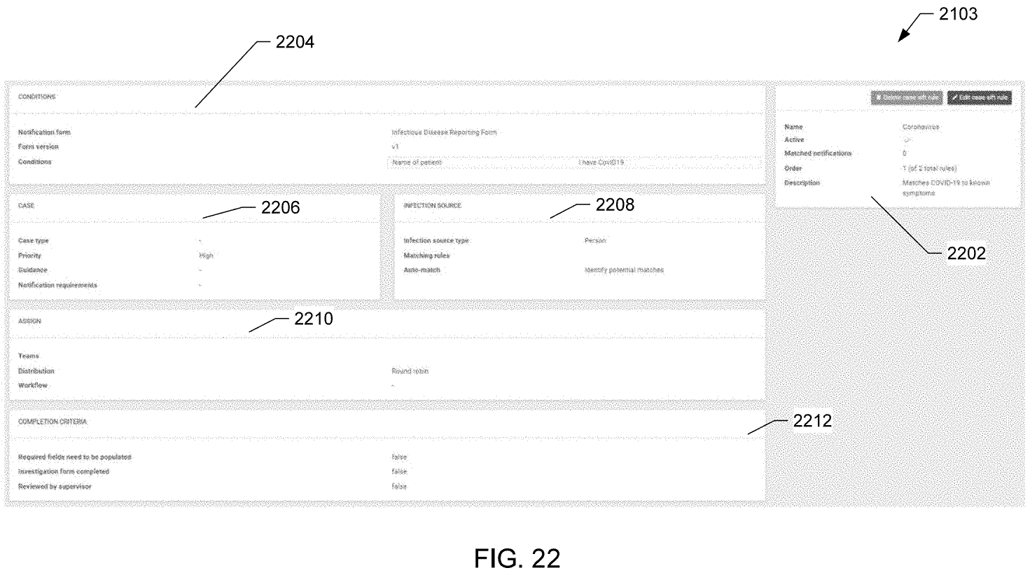

The example procedure 2100 of FIG. 21 begins when the outbreak management server 102 receives an indication to create a new case and/or cluster rule (block 2102). In response to the indication, the outbreak management server 102 displays a user interface 2103 that includes fields specifying a case and/or cluster rule. FIG. 22 is a diagram of the user interface 2103 for specifying a case and/or cluster rule, according to an example embodiment of the present disclosure. The user interface 2103 includes fields for a description 2202, conditions for the satisfying the rule 2204, case information for the rule 2206, an infection source for the rule 2208, assignment criteria 2210, and/or completion criteria 2212. In other embodiments the user interface 2103 may include fewer or additional fields.

The outbreak management server 102 prompts a user to complete the description section 2202 (block 2104). The description section 2202 includes fields for entry of a rule name, a summary of the rule, and a rule activation flag. When a rule is flagged as being inactive, the outbreak management server 102 is configured to skip the rule when assessing new patient medical data/patient data.

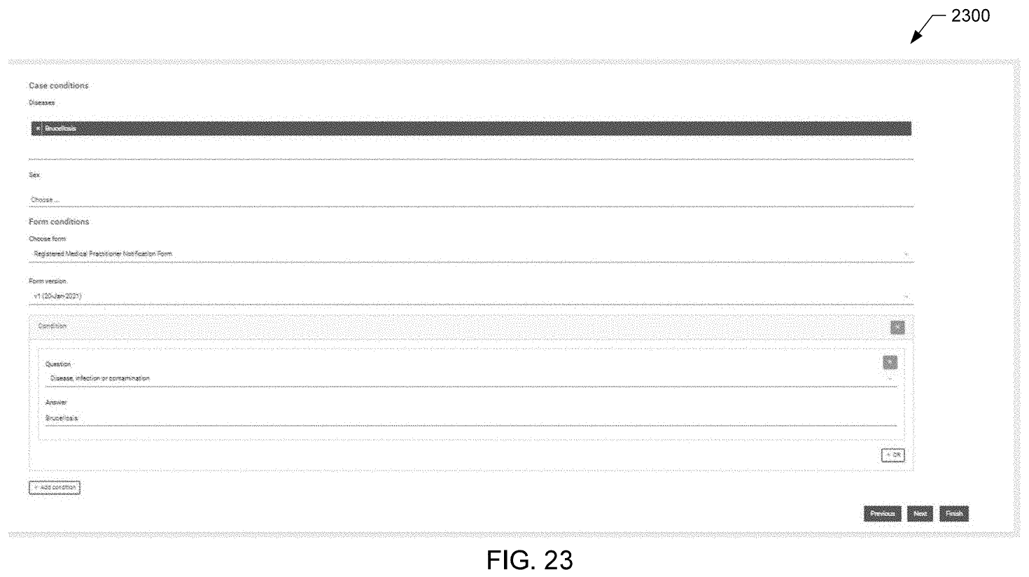

The outbreak management server 102 next prompts a user to complete the condition section 2204 by providing condition qualifiers (block 2106). FIG. 23 is a diagram of a user interface 2300 for entering condition information, according to an example embodiment of the present disclosure. As shown, conditions permit a user to set rules that define when a particular rule activates. The conditions are based on patient data 206, patient medical data 107, and/or information stored in a graph database structure for a patient. In some embodiments, the condition section 2204 may include a prompt for a catch-all rule to ensure each case and/or cluster receives an assignment.

To add a condition, the user interface 2300 includes a disease selector. Selection of this option enables a user to analyze a disease associated with a case. The user interface 2300 also includes a sex selector and a form selector. The sex selector enables a user to have a sex of a patient considered in analyzing a case. The form selector enables a user to use patient-responses to clinical questions be used in analyzing a case. Selection of the form selector may cause a drop-down list of form questions to be displayed. After a user selects a question, such as whether the disease was acquired through infection or contamination, the user interface 2300 includes an answer field. To satisfy the rule, an answer in the patient data 206, the patient medical data 107, and/or the information stored in a graph database structure has to match the answer specified for the rule. In some embodiments, the question/answer section may include symptoms, contact tracing information, and/or possible test results.

In some embodiments, the user interface 2300 may enable a user to add more than one answer option for a question. The user interface 2300 prompts a user to specify if both answers must be provided (AND) or if only one answer (OR) is needed to satisfy the rule. The user interface 2300 also permits a user to add multiple conditions for a rule.

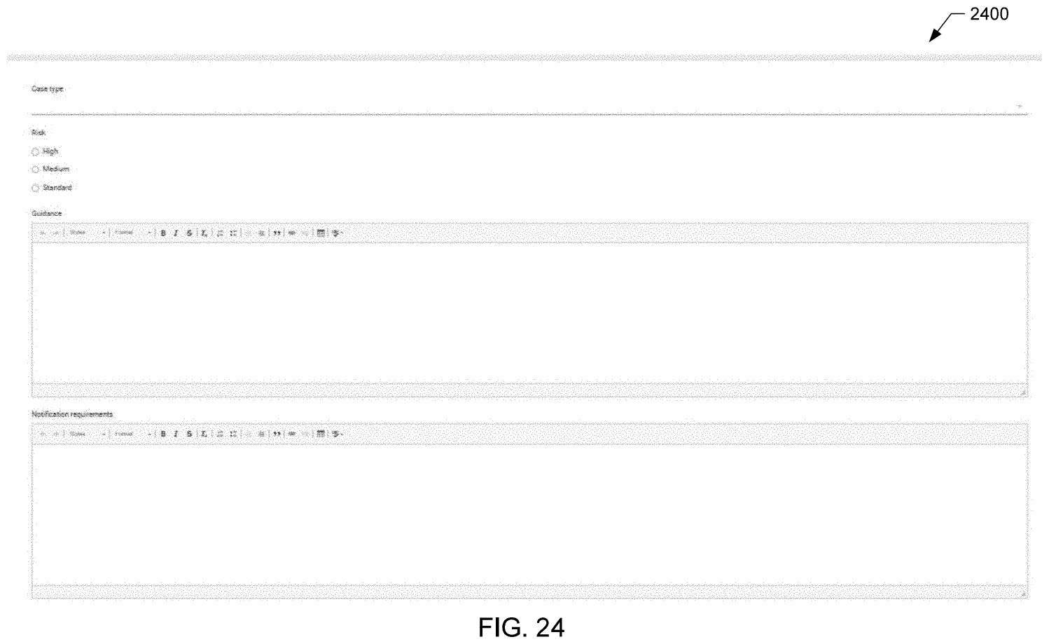

Returning to FIG. 21, the outbreak management server 102 is configured to prompt a user to enter the case information for the rule 2206 (block 2108). The case information defines aspects of the case that are created when patient data 206, patient medical data 107, and/or information stored in a graph database structure matches a rule. FIG. 24 shows a user interface 2400 provided by the outbreak management server 102 for configuring case information for population into the user interface 2103 of FIG. 22, according to an example embodiment of the present disclosure. The user interface 2400 includes a field for a case type sector, which is used to group cases together that are similar for forming clusters.

The user interface 2400 also includes a risk field, with options for ‘Standard’, ‘Medium’, or ‘High’. The risk information is used to communicate an importance of the case to other clinicians. The user interface 2400 further includes guidance and notification fields. The guidance field prompts a user to enter guidance information, which includes text that is added to a case following satisfaction of the rule. The text can be used to provide guidance on activities that may be carried out in a particular case, such as suggesting the need for contact tracing. The notification field prompts a user to enter notification information, which includes identifying one or more individuals and/or organizations that should be notified when the rule is satisfied. The notifications serve to inform clinicians of a new instance of a disease. Together, the guidance and notification fields specify who should be notified and the information needed for the notification when a particular rule is satisfied. In some embodiments, the user interface 2400 may include an option to add an investigation form to the case when the rule is satisfied.

The outbreak management server 102 is also configured to prompt a user for the infection source rule 2208 defined using the user interface 2103 (block 2110). This field prompts a user for an infection source type (e.g., person, animal, etc.), one or more matching rules for the infection source, and an indication if the outbreak management server 102 is to perform an auto-match or prompt a clinician to perform a manual match.

As shown in FIG. 21, the outbreak management server 102 prompts a user for the assignment criteria 2210 (block 2112). The assignment criteria 2210 specify rules or conditions as to how cases are distributed to clinician and other team members. The assignment criteria 2210 may include one or more fields to select a particular team and/or individual. Further, the assignment criteria 2210 include one or more distribution options as to how a new case is assigned and/or shared among the selected team/individual(s). The distributions options may include a random distribution, an assignment based on which team/individual has a fewest number of cases, and/or an assignment based on a care area or a treatment location.

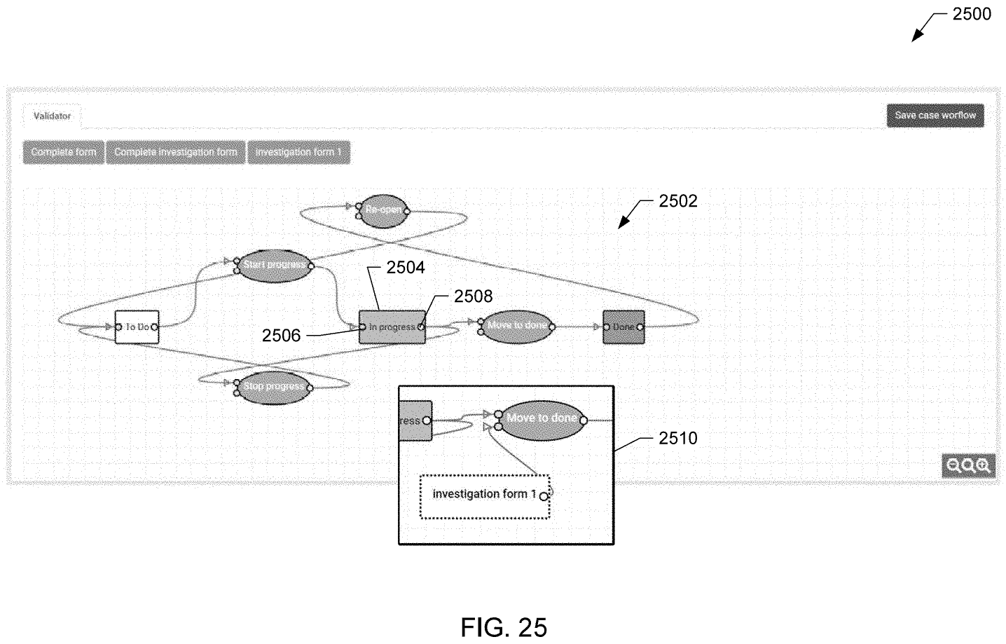

The assignment criteria 2210 may also enable a user to select and/or create a workflow for the case. In some instances, cases and/or diseases may have a one or more workflows that specify how a clinician is to treat a patient associated with the case and/or process the case. As discussed below in connection with FIG. 25, workflows identify stages of a case that must be satisfied and/or conditions of case that must be satisfied to process a case. It should be noted that a workflow may not necessarily correspond to successful treatment of a case, but at least initial classification and/or confirmation of a case as part of an outbreak.

After assignment, the outbreak management server 102 prompts a user for the completion criteria 2212 of the user interface 2103 (block 2114). The completion criteria 2212 may correspond to logic gates in the workflow, such as required fields to be populated for case entry, completion of certain forms, and/or a completion of a review by a supervisor. In some embodiments, the completion criteria 2212 may be automatically populated by a selected workflow. In other embodiments, selection of the completion criteria 2212 causes the outbreak management server 102 to automatically create a workflow using the information provided in the fields to define logic gates for the workflow.

The example procedure 2100 of FIG. 21 continues by the outbreak management server 102 receiving an indication that a case and/or cluster rule definition is complete (block 2116). The outbreak management server 102 uses the information entered into the user interface 2103 to create a case and/or cluster rule 2117, with the above information being used to create conditions to satisfying the rule, rule workflows, and/or actions to be carried out when the rule is satisfied. The outbreak management server 102 then stores the newly created case and/or cluster rule 2117 to the memory device 120 as a data structure or file (block 2118). As discussed below, the case and/or cluster rule 2117 is then used by the outbreak management server 102 for outbreak classification regarding newly received (and/or already received) patient data 206, patient medical data 107, and/or information stored in a graph database structure. After creation of the case and/or cluster rule 2117, the example procedure 2100 may return to block 2102 to create and/or modify another case and/or cluster rule.

As mentioned above, the user interface 2103 for the creation of a case and/or cluster rule 2117 enables one or more workflows to be selected. In some instances, the workflow may include a default workflow, which provides for transitions between three statuses (To do′, ‘In Progress’, and ‘Done’) for handing a case. In other instances, the workflows may include a custom workflow. FIG. 25 is a diagram of a user interface 2500 for a custom workflow 2502, according to an example embodiment of the present disclosure. In the illustrated example, the custom workflow 2502 includes a modification of a default workflow, with actions added for ‘Start Progress’, ‘Stop Progress’, ‘Re-Open’, and ‘Move to Done’. Each element or object of the workflow 2502 corresponds to an action to be performed. To make connections between the elements, the user interface 2500 is configured to enable a user to drag a line from an input of an object to an output of another object. For example, the ‘In Progress’ object 2504 includes an input 2506 and an output 2508. The user interface 2500 enables a user to specify a number of inputs/outputs for an object in addition to logic or other operations each object is to perform.

The custom workflow 2502 includes connections that enable cases to travel back through a workflow based on case progress. The custom workflow 2502 also includes a control (e.g., a validator) that defines workflow progression through the objects based on completion of form(s) and/or entry of certain data entry/collection for a case. For example, in the illustrated embodiment, a user has made an edit 2510 to the workflow 2502. The edit includes adding an input to the ‘Move to Done’ object and adding an ‘Investigative Form 1’ object. Before the workflow 2502 is deemed as being completed, the ‘Move to Done’ object has to be completed by receiving a complete ‘Investigative Form 1’. This workflow configuration ensures the same information is collected consistently for each case before the case can be designated as being complete.

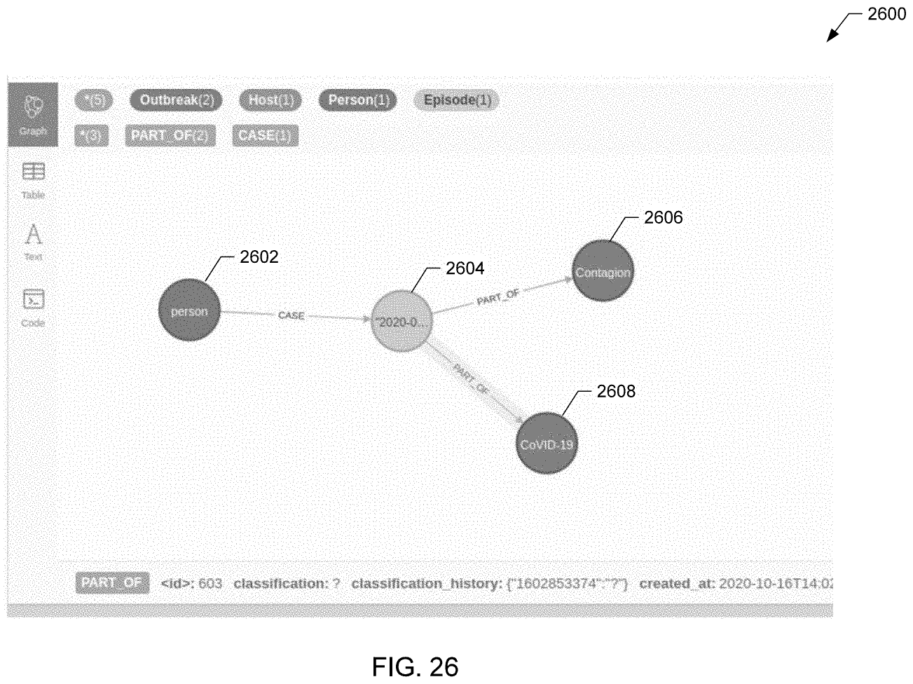

FIG. 26 is a diagram of a case/cluster user interface 2600 provided by the outbreak management server 102 for display in the application 302 of the user device 126, according to an example embodiment of the present disclosure. In the illustrated example, a person node 2602 is connected to an episode node 2604 via a ‘case’ link. A case is represented by the episode node 2604. The episode node 2604 is linked to a first outbreak node 2606 via a ‘part of’ link and a second outbreak node 2608 via another ‘part of’ link. Clusters are represented by outbreak nodes. The user interface 2600 accordingly provides a graphical relationship between an infection source (i.e., the person node 2602), a case (i.e., the episode node 2604), and clusters (i.e., the outbreak nodes 2606 and 2608). As shown, a host (i.e., the person node 2602) may have any number of cases (i.e., the episode node 2604), which may be part of one or more clusters (i.e., the outbreak nodes 2606 and 2608). In the illustrated example, a case may be associated with the COVID-19 outbreak in addition to a more general contagion outbreak. A case classification and its history, in this embodiment, are recorded as attribute information inside the ‘part of’ link or relationship. A case classification (e.g., ‘Probable’, ‘Possible’, ‘Confirmed’, etc.) is determined or assigned based on the outbreak definition, as discussed above in connection with FIG. 8.

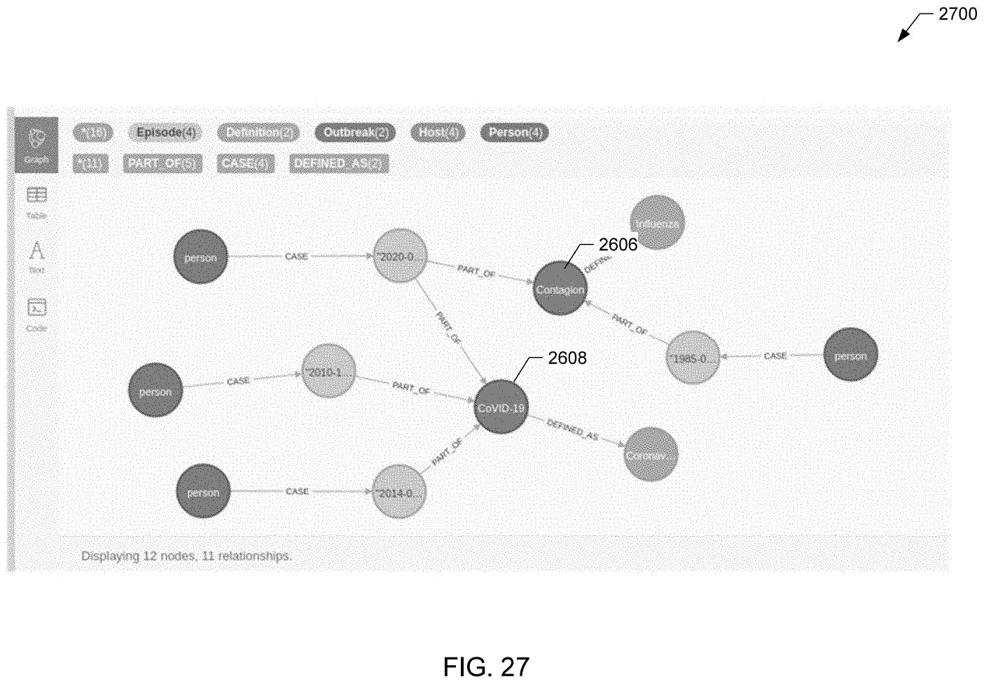

FIG. 27 is a diagram of another case/cluster user interface 2700 provided by the outbreak management server 102 for display in the application 302 of the user device 126, according to an example embodiment of the present disclosure. This example shows a larger group of infection sources liked to the outbreak nodes 2606 and 2608 via respective episode nodes. As additional infection sources are added, the example outbreak management server 102 may classify at least one of the outbreak nodes 2606 and 2608 as a confirmed outbreak. The use of the graph database accordingly enables connected sources of a disease to be visually and/or programmatically connected for analysis.

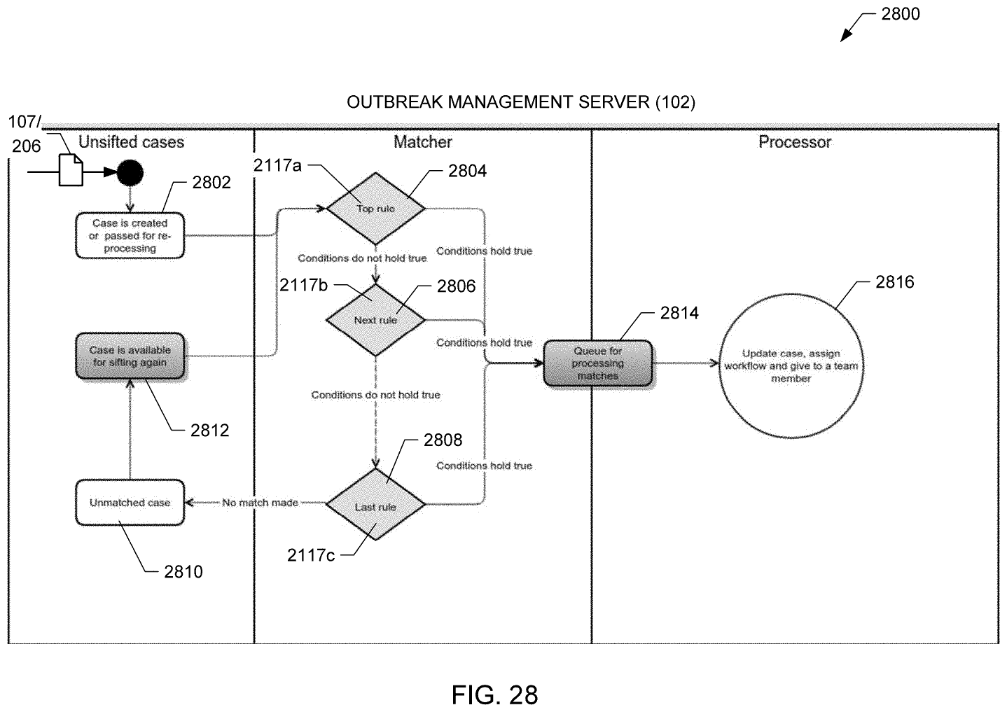

FIG. 28 illustrates a flow diagram showing an example procedure 2800 for comparing one or more cases to one or more case and/or cluster rules 2117, according to an example embodiment of the present disclosure. Although the procedure 2800 is described with reference to the flow diagrams illustrated in FIG. 28, it should be appreciated that many other methods of performing the steps associated with the procedure 2800 may be used. For example, the order of many of the blocks may be changed, certain blocks may be combined with other blocks, and many of the blocks described are optional. Further, the actions described in the procedure 2800 may be performed among multiple devices including, for example the outbreak management server 102, the user device 126, the clinician device 114, the HIS 104, and/or the servers 204 of FIGS. 1 to 3. Further, the procedure 2800 may be illustrative of the instructions 128 of FIG. 4.

The example procedure 2800 begins when patient medical data 107, patient data 206, and/or data from a graph database structure is received in the outbreak management server 102 as an un-sifted case (block 2802). In some embodiments, the outbreak management server 102 is configured to create an infection source node and/or an episode node to create a case for comparison to the one or more rules 2117. In other examples, the outbreak management server 102 may process previously un-sifted cases through the example procedure 2800. Further, in some embodiments, the outbreak management server 102 is configured to create a new case for sifting based on a notification of a disease case and/or cluster. The notification is based on the data 107 and/or 206, and may include data entered manually into completed electronic forms, a bulk upload via CSV format files, an import of laboratory identified disease results, manual creation of a case, manual creation of a cluster, and/or detection of a case or cluster within the data 107 and/or 206.

As shown, the outbreak management server 102 iterates through the rules 2117a to 2117c, for example, one rule at a time in a priority order for un-sifted cases. The rules 2117a to 2117c may be arranged or sequenced by an administrator and/or the outbreak management server 102 based on a risk designation, where higher risk rules are ordered first. In some instances, newer rules are placed at a top of the hierarchy. In some embodiments, the outbreak management server 102 periodically checks (e.g., every minute) for un-sifted cases and/or data 107, 206 for matching to the rules 2117. In the illustrative example, the outbreak management server 102 sequentially compares a hierarchy of rules 2117a, 2117b, and 2117c to information within a case (blocks 2804, 2806, and 2808). The outbreak management server 102 performs a cypher query between a rule 2117 and the case information to determine if the rule is satisfied. The cypher query may determine, for example, if the case (including any related forms and infection source) matches one or more conditions within a rule.

If the conditions of one rule are not satisfied or hold true, the outbreak management server 102 proceeds to the next rule 2117. If a case fails to satisfy any rule, the outbreak management server 102 causes the case to be stored to the memory device 120 as an unmatched case (block 2810). Alternatively, a catch-all rule 2117 may be placed last in the hierarchy to ensure all cases are assigned. At that point, the un-sifted case is available to be re-processed if case data is updated/changed and/or if a rule is modified or a new rule is added (block 2812). In some embodiments, a matched case may be re-sifted through the rules 2117 when information related to the case classification changes.

If there is a match to a rule, the outbreak management server 102 queues the case for processing matches (block 2814). It should be appreciated that placement of a case in a queue enables background machine resources to handle the subsequent processing of multiple queued cases in parallel or batches. This may be beneficial during times of high system load to give the outbreak management server 102 more flexibility regarding how cases are processed without overloading the system.

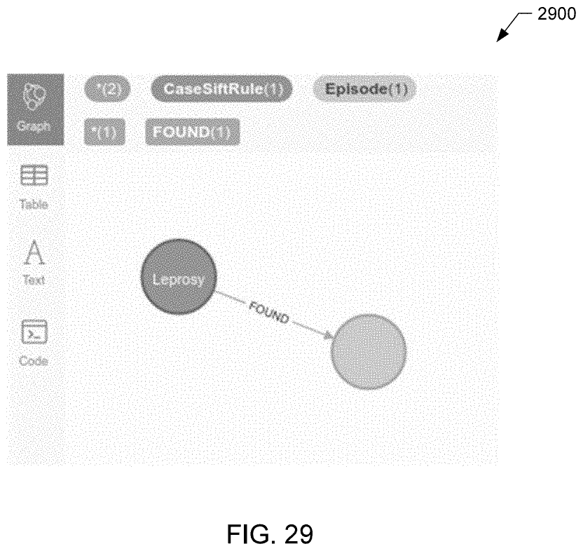

As shown in FIG. 28, the satisfaction of a rule causes the outbreak management server 102 to translate a case into work for users of a medical system (block 2816). The matching of a rule also causes the outbreak management server 102 to add a ‘found’ link or relationship between a node for the rule and the episode node in the graph database, as shown in user interface 2900 of FIG. 29. This illustration shows that each rule 2117 has a node in a graph database. The association of a case (via the episode node) to the rule 2117 prevents the outbreak management server 102 from associating the same episode to another rule with a lower priority in the hierarchy. Removal of the ‘found’ relation causes the case to be placed into the block 2812 for re-processing.

In addition to updating the graph database, the outbreak management server 102 may modify the case by applying a workflow from a satisfied rule, assigning the case to a clinician or team of clinicians using assignments defined by the rule, and/or setting key case fields such as risk, guidance, and/or notifications requirements. In some embodiments, the outbreak management server 102 enables a user to modify a workflow rule for a specific infection source. For assigning a case, the outbreak management server 102 is configured to follow the assignment conditions specified by the rule 2117. This may include randomly assigning a case to a team and/or individual, selecting a team and/or individual with a fewest number of cases, assigning a case based on available capacity of a team/individual, assigning a case sequentially through an alphabetic list of individuals/teams, and/or selecting an individual and/or team based on care area or medical location relative to a location of the infection source (using a location/stay nodes, discussed above). The assignment of a team or individual causes the outbreak management server 102 to create a team/individual node that is linked to the episode node via an ‘assigned’ link in the graph database.

The example outbreak management server 102 is configured to use the information from a rule 2117 to determine if a case or cluster is associated with a confirmed outbreak. As shown in FIGS. 26 and 27, infection sources and corresponding episode nodes are linked to outbreak nodes. However, a few linkages may be determined by the outbreak management server 102 as a possible outbreak. The outbreak management server 102 may analyze trends overtime to determine how quickly new cases are added to each outbreak for defined geographic areas (forming clusters). If a number of cases are added to an outbreak node within a defined time period (e.g., one week, two weeks, etc.) over a defined geographic area, the outbreak management server 102 is configured to change a possible outbreak classification to a probable or confirmed outbreak classification. In some instances, the number of cases may be based on a population density of a geographic area. For example, the threshold may be 5 new cases per week per 1000 people in the population.

V. Activity Mapping Embodiment

As discussed above in connection with FIG. 8, a host node is associated with one or more locations, as determined through self-reporting or contact tracing. In some embodiments, the host node of the graph database structure includes an activity mapping feature or parameter. Activity mapping is a process of detailing the locations an individual has visited and the persons they have been in contact with during the visit. The objective of activity mapping is to provide data for identifying how an outbreak is being spread and who may be at risk of getting a disease. The activity map is added to a person or infection source (e.g., a record of a host node) rather than an individual case or cluster. Activities can therefore be documented at discrete times, but they all contribute to a single map.

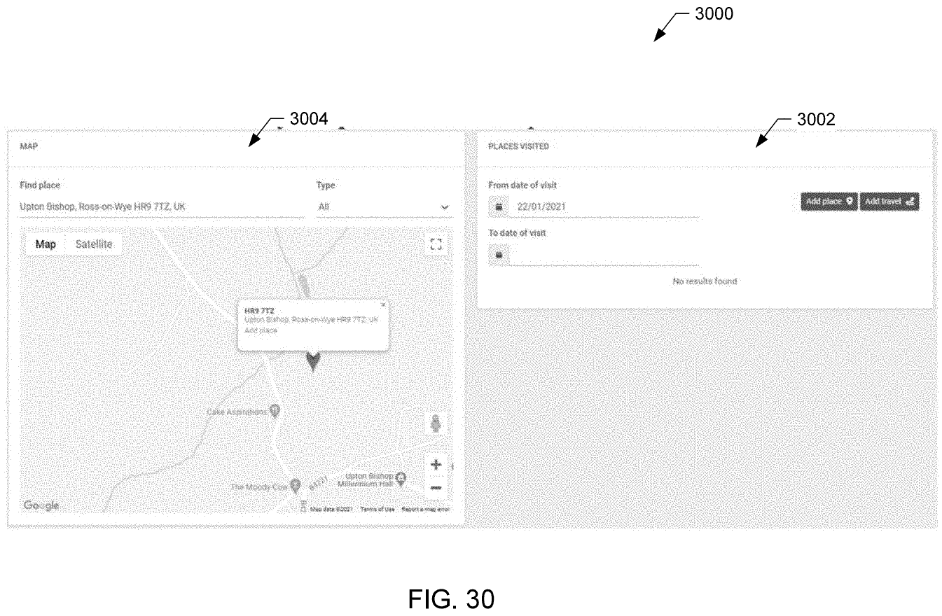

FIG. 30 is a diagram of a user interface 3000 for activity mapping, according to an example embodiment of the present disclosure. The user interface 3000 includes a section 3002 for adding an activity and/or location. Creation of an activity/location causes the outbreak management server 102 to link a location node to a host node via a stay node. To add a location, a user enters one date or a date range that a patient was at a particular location. The section 3002 may also include fields to enter a time, time range, or duration. A section 3004 includes a field for an address, establishment name, point-of-interest, etc. for a location. Entry of a location in the section 3004 causes the location to be displayed on a map. Confirmation of the location causes the location to be connected to the host node and displayed on the activity map, which is associated with the host node.

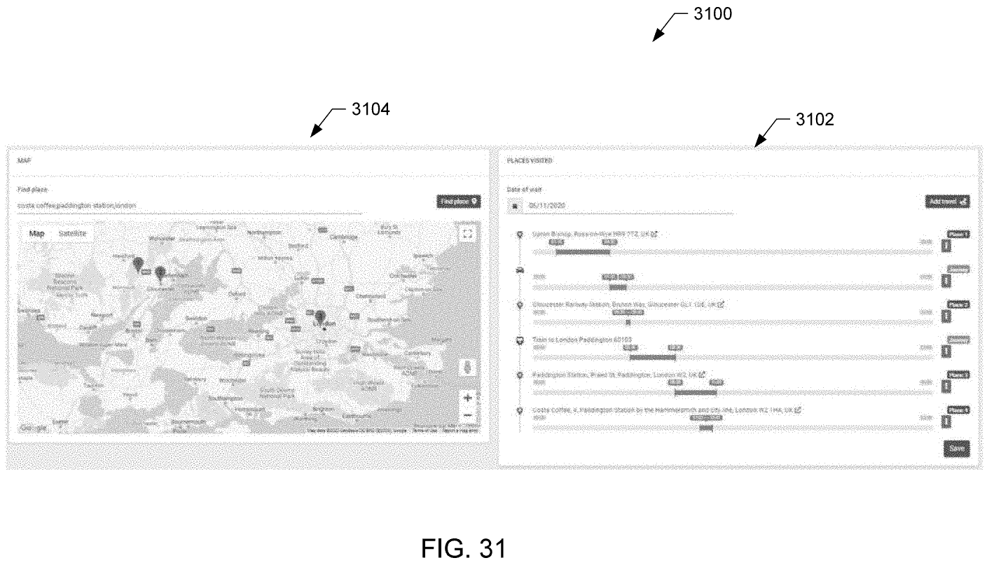

In some embodiments, the outbreak management server 102 is configured to enable a user to enter travel location information for a patient. FIG. 31 is a diagram of a user interface 3100 provided by the outbreak management server 102 for entering travel information. The travel information represents a journey between two locations. A section 3102 of the user interface 3100 includes fields for selecting a starting date, a time, a starting location, and a journey type (e.g., walking, car, train, plane, etc.). The section 3102 may include a duration slider to specify a time duration the patient was traveling. The section 3102 may also include a description field to describe or specify the type of journey. Further, the section 3102 may include a field that enables a user to select a flight number, train route number, bus route, etc. As shown, the section 3102 enables a user to specify more than one mode of transportation for a journey. The journey information is stored to the activity map in relation to the host node in addition to at least one location node. Further, the journey information is displayed on a map 3104.

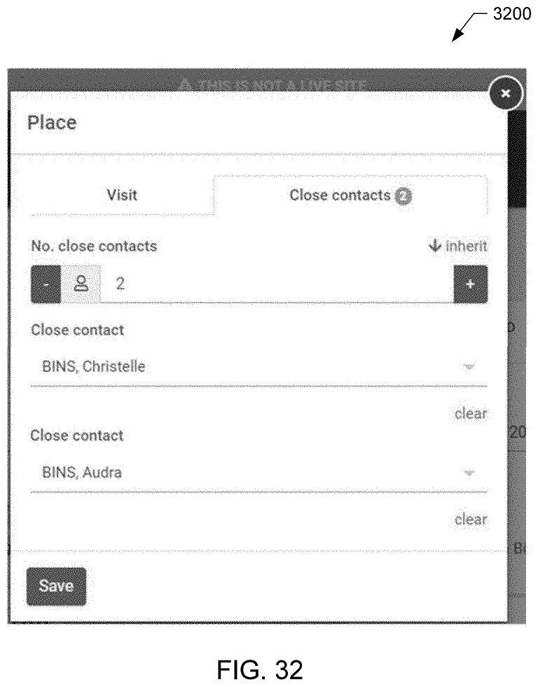

The example outbreak management server 102 is also configured to enable a user to document contacts of a patient in each location or journey. FIG. 32 is a diagram of a user interface 3200 for recording contact information for a location or journey (e.g., a place), according to an example embodiment of the present disclosure. Entry of contact information causes the outbreak management server 102 to create host and stay nodes for the contact individuals that are linked to the same location node as the patient host node. If there is an epidemiological link, the outbreak management server 102 may also create direct links between the patient host node and the contact nodes in the graph database structure.

Entry of contact information into the user interface 3200 also causes a contact entry to be created for the activity map 3104 for the entered location. For each location, the user interface 3200 provides fields to enter a number of close contacts and names of the close contacts. In some embodiments, the user interface 3200 may prompt a user for other information, such as a contact's email, phone number, home address, or other identifier needed to reach the contact.

The use of an activity map provides for visual contact tracing of a patient and known contacts. The outbreak management server 102 may combine data from activity maps from one or more patients (via corresponding host nodes) to identify geographic clusters of outbreaks and/or otherwise visually assess how a possible, probable, or confirmed outbreak is spreading overtime. In some embodiments, the aggregated map may have a time bar that shows host movement and locations of confirmed, probable, and possible cases over time.

### CONCLUSION

It will be appreciated that all of the disclosed methods and procedures described herein can be implemented using one or more computer programs or components. These components may be provided as a series of computer instructions on any conventional computer-readable medium, including RAM, ROM, flash memory, magnetic or optical disks, optical memory, or other storage media. The instructions may be configured to be executed by a processor, which when executing the series of computer instructions performs or facilitates the performance of all or part of the disclosed methods and procedures.

It should be understood that various changes and modifications to the example embodiments described herein will be apparent to those skilled in the art. Such changes and modifications can be made without departing from the spirit and scope of the present subject matter and without diminishing its intended advantages. It is therefore intended that such changes and modifications be covered by the appended claims.

It should be appreciated that 35 U.S.C. 112(f) or pre-AIA 35 U.S.C. 112, paragraph 6 is not intended to be invoked unless the terms “means” or “step” are explicitly recited in the claims. Accordingly, the claims are not meant to be limited to the corresponding structure, material, or actions described in the specification or equivalents thereof

The invention is claimed as follows:

1. An outbreak management server system comprising:
a memory device storing (i) case rules each specifying at least one condition for satisfying the respective case rule, and (ii) instructions therein, the instructions defining a graph database structure for disease outbreak tracking, the instructions specifying for a given host that:
a host node is created, the host node being associated with host parameters,
an episode node is connected to the host node via a ‘case’ link, the episode node being associated with episode parameters that are related to a disease classification of the host, and
a rule node is connected to the episode node via a ‘found’ link, the rule node being associated with a case workflow, an assignment, and a notification; and
an outbreak management server configured to:
receive patient data related to the host,
store at least some of the received patient data to the graph database at one or more parameters of at least one of the host node or the episode node based on contents of the at least some of the received patient data matching parameter definitions of the respective node,
compare parameters of the host node and the episode node to the conditions of the case rules, and
for a case rule having at least one condition that first matches the parameters of the host node and episode node, create a link between the rule node and the episode node, the linkage causing a case workflow of the case rule to be added for the episode node, the episode node to be assigned to an individual or a team, and a notification to be generated regarding association with the case rule.

2. The system of claim 1, wherein the case rules are arranged in an order or hierarchy, and
wherein the outbreak management server is further configured to sequentially compare the one or more conditions of each of the case rules to the parameters of the host node and episode node until there is a match to one case rule.

3. The system of claim 1, wherein the case workflow defines a sequence of steps specified by objects for obtaining documentation related to the case of the episode node or managing a disease associated with the episode node.

4. The system of claim 1, wherein the outbreak management server is further configured to use the assignment of the case rule to create a leakage between the episode node and a node of an individual or a team via an ‘assigned’ link.

5. The system of claim 1, wherein the notification identifies one or more individuals or organizations that should receive a message indicative of the case rule being satisfied for the host node and the episode node.

6. The system of claim 5, wherein the case rule includes guidance information for treating the case, the guidance information being included within the message that is related to the notification.

7. The system of claim 1, wherein each of the case rules are data structures or files that include the one or more conditions, assignment information, a link to the case workflow, and notification information.

8. The system of claim 1, wherein after the case workflow is completed, the outbreak management server is further configured to connect an outbreak node to the episode node via a ‘part of’ link to indicate that the host has become part of an outbreak of the disease when a result of the workflow is indicative that the case is associated with the outbreak, and
wherein the outbreak node is connected to a definition node via a ‘defined as’ link, the definition node specifying disease parameters of the disease that are related to the outbreak node.

9. The system of claim 8, wherein the disease parameters include at least one of a name of the disease, a background of the disease, a time/place related to the disease, clinical criteria for the disease, laboratory criteria for the disease, modes of transmission for the disease, criteria for determining a ‘suspected’ classification, criteria for determining a ‘probable’ classification, and criteria a determining the ‘confirmed’ classification.

10. The system of claim 1, wherein the server is further configured to connect the host node to the outbreak node via the episode node after determining at least some of the patient data matches at least some of the disease parameters of the definition node.

11. The system of claim 1, wherein the host parameters include at least one of a name of the host, a patient classification flag, a clinician classification flag, a person classification flag, an animal classification flag, a fomite classification flag, an object classification flag, patient demographic data, or patient medical data.

12. The system of claim 1, wherein the episode parameters include at least one of a start of a case number for the host, a ‘possible’ classification for the disease for the host, a ‘probable’ classification for the disease for the host, a ‘confirmed’ classification for the disease for the host, an immunization status of the host, an immunization type of the host, a flag indicative that the host acquired the disease in a medical facility, or death information related to the host.

13. The system of claim 1, wherein the outbreak management server is further configured to generate a case number for the respective episode parameter of the episode node after determining a ‘confirmed’ or a ‘probable’ classification for the host.

14. The system of claim 1, wherein the outbreak management server is further configured to:
receive at least one of a geographic location or a journey associated with the host node;
create an activity map that is related to the host node for the at least one geographic location or journey; and
create stay nodes linked to the host node via a ‘stayed’ link and location nodes linked to one of the stay nodes via an ‘in’ link for the at least one geographic location or journey.

15. An outbreak management method comprising:
storing, in a memory device, (i) case rules each specifying at least one condition for satisfying the respective case rule, and (ii) a graph database structure for disease outbreak tracking, the graph database structure configured, for a given host, such that:
when a host node is created, the host node is associated with host parameters,
when an episode node is connected to the host node via a ‘case’ link, the episode node is associated with episode parameters that are related to a disease classification of the host, and
when a rule node is connected to the episode node via a ‘found’ link, the rule node is associated with a case workflow, an assignment, and a notification;
receiving, in an outbreak management server, patient data related to the host;
storing, via the outbreak management server, at least some of the received patient data to the graph database at one or more parameters of at least one of the host node or the episode node based on contents of the at least some of the received patient data matching parameter definitions of the respective node;
comparing, via the outbreak management server, parameters of the host node and the episode node to the conditions of the case rules; and
for a case rule having at least one condition that first matches the parameters of the host node and episode node, creating, via the outbreak management server, a link between the rule node and the episode node, the linkage causing a case workflow of the case rule to be added for the episode node, the episode node to be assigned to an individual or a team, and a notification to be generated regarding association with the case rule.

16. The method of claim 15, wherein the case rules are arranged in an order or hierarchy, and
the method further comprises sequentially comparing, via the outbreak management server, the one or more conditions of each of the case rules to the parameters of the host node and episode node until there is a match to one case rule.

17. The method of claim 15, wherein the case workflow defines a sequence of steps specified by objects for obtaining documentation related to the case of the episode node or managing a disease associated with the episode node.

18. The method of claim 15, further comprising using, via the outbreak management server, the assignment of the case rule to create a leakage between the episode node and a node of an individual or a team via an ‘assigned’ link.

19. The method of claim 15, wherein the notification identifies one or more individuals or organizations that should receive a message indicative of the case rule being satisfied for the host node and the episode node, and
wherein the case rule includes guidance information for treating the case, the guidance information being included within the message that is related to the notification.

20. The method of claim 15, further comprising connecting, via the outbreak management server, an outbreak node to the episode node via a ‘part of’ link to indicate that the host has become part of an outbreak of the disease when a result of the workflow is indicative that the case is associated with the outbreak,
wherein the outbreak node is connected to a definition node via a ‘defined as’ link, the definition node specifying disease parameters of the disease that are related to the outbreak node.
